(19) United States
(12) Patent Application Publication
(10) Pub. No.: US 2004/0123334 A1
(43) Pub. Date: Jun. 24, 2004

(54) Methods of treating neurodegenerative diseases

(75) Inventors: Kun Ping Lu, Newton, MA (US); Tony R. Hunter, Del Mar, CA (US); Yih-Cherng Liou, Singapore (SG)

Correspondence Address: LAHIVE & COCKFIELD, LLP., 28 STATE STREET, BOSTON, MA, 02109, US

(73) Assignees: BETH ISRAEL DEACONESS MEDICAL CENTER, Boston, MA; The Salk Institute for Biological Studies, La Jolla, CA

(21) Appl. No.: 10/641,815

(22) Filed: Jan. 12, 2004

Related U.S. Application Data
(60) Provisional application No. 60/404,030, filed on Aug. 15, 2002.
(60) Provisional application No. 60/469,546, filed on May 8, 2003.

(51) Int. Cl. A01K 67/00; A61K 49/00

(52) U.S. Cl. 800/3; 424/9.2; 800/12

(57) ABSTRACT

An animal model for neurodegenerative disorders, e.g., a transgenic model in which the Pin1 gene is misexpressed is described. The animal is useful for identifying and monitoring treatments and agents for a number of neurodegenerative disorders. Accordingly, also provided are methods for preventing, treating and/or delaying the onset of neurodegenerative disorders by administering to a subject in need thereof and agent that increases Pin1 biological activity in neuronal tissues and fluids.

### RELATED APPLICATIONS

This application claims priority to U.S. Provisional Patent Application No. 60/469,546, filed on May 8, 2003 and to U.S. Provisional application Serial No. 60/404,030, filed Aug. 15, 2002, the entire contents of which are hereby incorporated by reference.

### GOVERNMENT FUNDING

The work was supported by NIH grants GM58556 and AG17870.

### BACKGROUND OF THE INVENTION

A neuropathological hallmark in Alzheimer's disease, Pick disease, progressive supranuclear palsy, corticobasal degeneration and frontotemporal dementia and parkinsonism linked to chromosome 17 (FTDP-17) is the neurofibrillary tangles, whose main component is the microtubule-associated protein tau (1-3). Tau is hyperphosphorylated in tangles (4-6), and phosphorylation of tau causes loss of its ability to bind microtubules and promote microtubule assembly. Importantly, tau point mutations can cause FTDP-17. Furthermore, transgenic overexpression of the tau mutants in mice mimics the features of human tauopathies (12-16). These results prove that tau dysfunction can directly cause neurodegeneration.

Interestingly, tangle formation in Alzheimer's disease is preceded by increased phosphorylation of tau and other proteins on certain serine or threonine residues preceding proline (pSer/Thr-Pro) (17-19). Many of such pSer/Thr-Pro motifs are also known as MPM-2 epitopes due to their recognition by MPM-2, a mitosis-specific, phosphorylation-dependent monoclonal antibody (mAb). In fact, increased phosphorylation on MPM-2 epitopes is a prominent and common feature in Alzheimer's disease (AD) and related neurodegenerative disorders including frontotemporal dementia, Parkinsonism linked to chromosome 17 (FTDP-17), Down Syndrome, coricobasal degeneration, progressive supranuclear palsy and Picks disease. Significantly, pSer/Thr-Pro motifs in proteins exist in the two completely distinct cis and trans conformations, whose conversion is significantly reduced upon phosphorylation, but is specifically catalyzed by the prolyl isomerase Pin1 (21-25). It has been established that via its N-terminal WW domain, Pin1 is targeted to MPM-2 epitopes in a defined subset of phosphoproteins, where its C-terminal isomerase domain specifically catalyzes the isomerization of pSer/Thr-Pro motifs to induce conformational changes. Such conformational changes following phosphorylation may have profound effects on catalytic activity, dephosphorylation, protein-protein interactions, subcellular location, and/or turnover of Pin1 substrate. Therefore, phosphorylation-dependent prolyl isomerization is a post-phosphorylation signaling mechanism that plays an important role in phosphorylation signaling (21).

Several in vitro results imply a possible involvement of Pin1 in neurodegenerative disease, i.e., Alzheimer's disease. Pin1 can directly bind phosphorylated tau and restore its ability to bind microtubules and promote microtubule assembly in vitro. Furthermore, Pin1 is required for efficient dephosphorylation of tau in vitro, because Pro-directed phosphatases such as tau phosphatase PP2A are conformation-specific, dephosphorylating only trans, but not cis, pSer/Thr-Pro motifs. The levels of soluble Pin1 have been shown to be depleted in brains of patients with Alzheimer's disease in U.S. Pat. No. 6,495,376. Finally, in contrast to many cancer tissues, where Pin1 is overexpressed, Pin1 is depleted in AD brains due to its high affinity with phosphorylated tau in the tangles (28).

### SUMMARY OF THE INVENTION

The present invention is based, at least in part, on the discovery that mice containing mutations in Pin1 demonstrate increased topologies associated with neurodegenerative diseases. Accordingly, it has been demonstrated that a depletion of Pin1 activity results in the degeneration of neural tissues associated with disease, and Pin1 mutant mice provide a novel model to test compounds that can be used to treat and/or prevent neurodegenerative disease.

In one aspect, the invention features a method for identifying an agent that is useful for the treatment of a neurodegenerative disorder. The method includes the steps of (a) administering an agent to an animal containing a mutation in a Pin1 gene, wherein the animal displays a neurodegenerative phenotype associated with the mutation; and (b) monitoring the neurodegenerative phenotype in the animal, wherein a decrease in the severity of the neurodegenerative phenotype is indicative that the agent is useful for treating a neurodegenerative disorder.

In another aspect, the invention features a method for identifying an agent that is useful for preventing or delaying the onset of a neurodegenerative disorder. This method includes the steps of (a) administering an agent to an animal containing a mutation in a Pin1 gene; and (b) monitoring the animal for a neurodegenerative phenotype associated with the mutation, wherein the absence or delay in the onset of the phenotype in the animal is indicative that the agent is useful for preventing or delaying onset of the neurodegenerative disorder.

In still another aspect, the invention features a method for identifying an agent that is useful for preventing or delaying the progression of a neurodegenerative disorder. This method includes the steps (a) administering an agent to an animal comprising a mutation in a Pin1 gene; and (b) monitoring the animal for a neurodegenerative phenotype associated with the mutation, wherein the absence or delay in the progression of the neurodegenerative phenotype is indicative that the agent is useful for preventing or delaying the progression of the neurodegenerative disorder.

The invention further features, a method of evaluating the efficacy of a treatment for a neurodegenerative disorder. This method includes the steps of (a) administering the treatment to a animal having a mutation in a Pin1 gene or a cell therefrom, wherein the animal displays a neurodegenerative phenotype associated with the mutation; and (b)determining the effect of the treatment on the neurodegenerative phenotype, thereby evaluating the efficacy of the treatment. The method may be performed in vivo or in vitro.

In certain embodiments, the neurodegenerative phenotypes monitored or examined in the methods of the invention include, but are not limited to, one or more of the following: an age-dependent neurological phenotype, a reduction in mobility, a reduction in vocalization, abnormal limb-clasping reflex, retinal atrophy inability to succeed in a hang test, an increased level of MPM-2 (e.g., MPM-2 epitopes), an increased level of neurofibril tangles, increased tau phosphorylation, tau filament formation, abnormal neuronal morphology, lysosomal abnormalities, neuronal degeneration, and gliosis.

In other embodiments the animal used in the methods of the invention, which is preferably a transgenic animal, is a mammal, e.g., a non-human primate or a swine (e.g., miniature swine, a monkey), a goat or a rodent (e.g., rat, hamster or mouse). Preferably, the animal is a mouse.

In other embodiments, expression of the mutation in the Pin1 gene results in a decrease in gene expression as compared to the wild-type animal. For example, the levels of Pin1 protein can be suppressed, at least, 50%, 60%, 70%, 80%, 90% or 100% as compared to the wild-type animal. In one preferred embodiment, the Pin1 gene is disrupted by removal of DNA encoding all or part of the protein. More preferably, the animal is homozygous for the disrupted gene. In still other preferred embodiments, the animal contains a conditional mutation in the Pin1 gene such that expression of the gene is decreased under particular conditions, or in specific cell types (e.g., neurons, preferably, brain neurons).

In still other embodiments, animals used in the methods of the invention can also contain a mutation in a second gene associated with a neurodegenerative phenotype. Non-limiting examples of second mutations include mutation that result in overexpression of APP, tau and/or presenilin. In additional embodiments, the animal can contain mutations in one or more genes associated with neurodegenerative disorders.

In another aspect, the invention also features methods of treating, preventing, delaying the onset or progression of a neurodegenerative disorder in a subject (e.g., mammal, preferably, a human) by administering an agent identified using the methods of the invention. In preferred embodiments, neurodegenerative disorders include, but are not limited to Alzheimer's disease, Pick disease, progressive supranuclear palsy, corticobasal degeneration, frontaltemporal dementia and Parkinsonism linked to chromosome 17.

In specific embodiments, the invention provides methods of treating or preventing the onset or progression of a neurodegenerative disease by upregulating the biological activity of Pin1. For example, Pin1 biological activity can be increased by a number of methods including, but not limited to, decreasing the rate of degradation of Pin1, decreasing the phosphorylation of Pin1, increasing the catalytic activity of Pin1, and/or increasing the expression of Pin1, (e.g., by gene therapy).

In particular embodiments, gene therapy methods are provided in which a nucleic acid encoding Pin1, or portion thereof (e.g., the isomerase domain) is provided to the nervous system such that Pin1 biological activity is increased.

In another embodiment, the invention provides a method of treating subject suffering from a neurodegenerative disease or disorder characterized by a decrease in Pin1 wherein a subject is administered an agent that increases the biological activity of Pin1.

In another aspect, the invention provides a method of treating a subject with a neurodegenerative disease, comprising determining the level of phosphorylated Tau in a biological sample, wherein an increased level of phosphorylated Tau in the sample is indicative of a Pin1-associated neurodegenerative disease, and administering to the subject an agent that increases the biological activity of Pin1.

In another aspect, the invention provides a method of diagnosing a subject with a neurodegenerative disease comprising measuring the amount of Pin1 in a neurological sample, e.g., spinal fluid, or brain tissue.

Other features and advantages of the invention will be apparent from the following detailed description, and from the claims.

### BRIEF DESCRIPTION OF THE DRAWINGS

FIG. 1 Inverse correlation of Pin1 expression with the predicted neuronal vulnerability and actual neurofibrillary degeneration in AD a, b. Normal (a) or AD (b) hippocampus sections were immunostained with anti-Pin1 antibodies (a) or with Pin1 antibodies (yellow) and AT8 (purple) (b). c. The relationship between Pin1 immunoreactivity and NFTs in AD hippocampus. ˜1000 pyramidal neurons were randomly selected and evaluated for AT8-positive or -negative NFTs, and Pin1 light (low) or intense (high) immunoreactivity. d. The relationship between Pin1 immunoreactivity and NFTs in tangle-rich CA1 region detected by Pin1 antibodies (red) and AT8 (green), respectively. Pin1 expression in most tangle-bearing neurons (arrowheads) was low than that in tangle-free neurons (arrows).

FIG. 2 Age-dependent motor and behavioral deficits in Pin1^{-/-} mice. a. Abnormal limb-clasping reflexes in old Pin1^{-/-} mice. When lifted by the tail, young WT and Pin1^{-/-} mice (2-3 months) and old WT mice (9-14 months) acted normally by extended their hind limbs and body, but old Pin1^{-/-} mice flexed their legs to the trunk or tightened the back limbs to their bodies. b. Hunched postures displayed by old Pin1^{-/-}, but neither young Pin1^{-/-} nor all WTmice. c. Age-dependent motor disturbance. Over 10 mice at different ages were placed onto a hanging bar and the percentage of the mice that fell off during one-minute test period was recorded. **, p<0.01.

FIG. 3 Age-dependent neuronal degeneration and loss in Pin1^{-/-} mice. a, b. Matched parietal cortex from WT Pin1^{-/-} mice were stained for NeuN (a), followed by counting neurons (b). *, p<0.01 c-h. Ultrastructure and AT8 immuno-gold labeling of degenerative neurons in Pin1^{-/-} hippocampus. c. WT neuron not labeled by AT8; d. Pin1^{-/-} neuron labeled with AT8 immuno-gold (sharp arrows) and containing dark and degenerated organelles (arrows) in the cytoplasm and near the nucleus (n); e. autophagic vacuole (arrow) near the nuclear membrane (arrowheads); f. Axon degeneration (arrow); g, h. neuron containing multiple compact and radiating structures (arrows) (g) composed of radiating filament-like structures (arrow) (h). Scale bars: c, d. 50 μm; e, 133 μm; f, 1 μm; 50 μm; g, 50 μm; h, 355 nm.

FIG. 4 MPM-2 induction, tau hyperphosphorylation, NFT-specific conformations and reduced phosphatase activity toward pSer/Thr-Pro motif in Pin1^{-/-} brain. a, Soluble brain extracts from age-matched old WT and Pin1^{-/-} mice were immnunoblotted with MPM-2. b-d. Sarcosyl-insoluble fractions (b, d) were prepared from age-matched WT and Pin1^{-/-} brains, followed by immunoblot with total tau mAb Tau-5 (b) or various phosphorylation- and/or NFT-specific tau mAbs (d). e. Age-dependent induction of the TG3 epitope. c and f. Sarcosyl-insoluble tau was pretreated with phosphatases before subjecting to immunoblot. Arrows point to 68 kDa tau. g. Subcellular localization of MPM-2 epitopes, tau phosphoepitopes and NFT-conformation epitopes in Pin1^{-/-} neurons, whereas no positive staining in WT neurons, as determined by immunostaining. h Reduced phosphatase activity towards pSer/Thr-Pro motifs, but not towards a non-pSer/Thr-Pro motif in Pin1^{-/-} brain lysates, as assayed using indicated substrates.

FIG. 5 NFT-like pathologies and tau filaments in Pin1^{-/-} neurons. a-d. Positive Gallyas silver staining of Pin1^{-/-} neurons. Different regions of old WT (a) or Pin1^{-/-} (b-d) brains were subjected to Gallyas silver staining. e-h. Positive thioflavin-S staining of Pin1^{-/-} neurons. Different regions of old WT (e, g) or Pin1^{-/-} (f, h) brains were subjected to thioflavin-S staining. i-j. Tau filaments isolated from Pin1^{-/-} brains. Sarcosyl-insoluble extracts were prepared from old Pin1^{-/-} (i) and WT (j) mice and examined under EM. k-l. Phosphorylated tau in the filaments. The sarcosyl-insoluble extracts were subjected to immunogold staining using AT8 (k) or AT180 (l), followed by EM.

### DETAILED DESCRIPTION

Neuropathological hallmarks of Alzheimer's disease (AD) and other tauopathies are senileplaques and/or neurofibrillary tangles (1-4). Although many mouse models have been created by overexpression of specific proteins including APP, presenilin and/or tau¹⁻¹⁰, no such a model has been generated by gene knockout. Phosphorylation of tau and other proteins on serine or threonine residues preceding proline (pSer/Thr-Pro) appears to precede tangle formation and neurodegeneration in AD (11-14). Interestingly, pSer/Thr-Pro motifs exist in two distinct conformations, whose conversion in certain proteins is catalyzed by the prolyl isomerase Pin1 (15-17). Pin1 activity can restore the conformation and function of phosphorylated tau directly and indirectly via promoting its dephosphorylation, suggesting an involvement of Pin1 in neurodegeneration (14, 18, 19). However, genetic evidence is lacking. Here we show that Pin1 expression inversely correlates with the predicted neuronal vulnerability and actual neurofibrillary degeneration in AD. Moreover, Pin1 is the first gene whose knockout in mice causes progressive age-dependent neuropathy characterized by motor and behavioral deficits, tau hyperphosphorylation, tau filament formation and neuronal degeneration. Thus, Pin1 plays a pivotal role in protecting against age-dependent neurodegeneration and provides a new insight into the pathogenesis and treatment of AD and other tauopathies.

The present invention is based, at least in part, on the generation of animals which are homozygous for a null mutation in the Pin1 gene and the observation that these animals display neurodegenerative phenotypes. The invention is further based on the observation that Pin1 expression and/or activity inversely correlates with neurofibillary degeneration in neurodegenerative diseases, e.g., Alzheimer's disease. Accordingly, the invention features methods that utilize a non-human animal in which the gene encoding the Pin1 protein is misexpressed as a model for neurodegenerative disorders. In preferred embodiments the animal, is a transgenic animal. Based on the results using the mouse model of the invention, methods of treating or preventing the onset or progression of a neurodegenerative disease are also provided.

As used herein, a “transgenic animal” includes an animal, e.g., a non-human mammal, e.g., a swine, a monkey, a goat, or a rodent, e.g., a mouse, in which one or more, and preferably essentially all, of the cells of the animal include a transgene. The transgene is introduced into the cell, directly or indirectly by introduction into a precursor of the cell, e.g., by microinjection, transfection or infection, e.g., by infection with a recombinant virus. The term genetic manipulation includes the introduction of a recombinant DNA molecule. This molecule may be integrated within a chromosome, or it may be extrachromosomally replicating DNA.

As used herein, the term “rodent” refers to all members of the phylogenetic order Rodentia.

As used herein, the term “misexpression” includes a non-wild type pattern of gene expression. Expression as used herein includes transcriptional, post transcriptional, e.g., mRNA stability, translational, and post translational stages. Misexpression includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus. Misexpression includes any expression from a transgenic nucleic acid. Misexpression includes the lack or non-expression of a gene or transgene, e.g., that can be induced by a deletion of all or part of the gene or its control sequences.

As used herein, the term “knockout” refers to an animal or cells therefrom, in which the insertion of a transgene disrupts an endogenous gene in the animal or cell therefrom. This disruption can essentially eliminate Pin1 in the animal or cell.

In preferred embodiments, misexpression of the gene encoding the PIN1 protein is caused by disruption of the PIN1 gene. For example, the PIN1 gene can be disrupted through removal of DNA encoding all or part of the protein.

In preferred embodiments, the animal can be heterozygous or homozygous for a misexpressed PIN1 gene, e.g., it can be a transgenic animal heterozygous or homozygous for a PIN1 transgene.

In preferred embodiments, the animal is a transgenic mouse with a transgenic disruption of the PIN1 gene, preferably an insertion or deletion, which inactivates the gene product. The nucleotide sequence of the wild type PIN1 is known in the art and described in, for example, U.S. Pat. No. 5,972,697, the contents of which are incorporated herein by reference. Preferred embodiments also include animals in which one or more genes, in addition to Pin1, are misexpressed. For example, the animals used in the methods of the invention can also contain other mutations associated with neurodegenerative diseases, e.g., mutations in APP, tau and/or preselin (59-63).

As used herein, the term “marker sequence” refers to a nucleic acid molecule that (a) is used as part of a nucleic acid construct (e.g., the targeting construct) to disrupt the expression of the gene of interest (e.g., the PIN1 gene) and (b) is used to identify those cells that have incorporated the targeting construct into their genome. For example, the marker sequence can be a sequence encoding a protein which confers a detectable trait on the cell, such as an antibiotic resistance gene, e.g., neomycin resistance gene, or an assayable enzyme not typically found in the cell, e.g., alkaline phosphatase, horseradish peroxidase, luciferase, beta-galactosidase and the like.

As used herein, “disruption of a gene” refers to a change in the gene sequence, e.g., a change in the coding region. Disruption includes: insertions, deletions, point mutations, and rearrangements, e.g., inversions. The disruption can occur in a region of the native PIN1 DNA sequence (e.g., one or more exons) and/or the promoter region of the gene so as to decrease or prevent expression of the gene in a cell as compared to the wild-type or naturally occurring sequence of the gene. The “disruption” can be induced by classical random mutation or by site directed methods. Disruptions can be transgenically introduced. The deletion of an entire gene is a disruption. Preferred disruptions reduce PIN1 levels to about 50% of wild type, in heterozygotes or essentially eliminate PIN1 in homozygotes.

The term “neurodegenerative” as used herein, is used to designate a group of disorders in which there is gradual, generally relentlessly progressive wasting away of structural elements of the nervous system. As used herein, the term “neurodegenerative phenotype” includes any parameter related to neurodegeneration, e.g., a reduction in mobility, a reduction in vocalization, abnormal limb-clasping reflex, retinal atrophy inability to succeed in a hang test, an increased level of MPM-2, an increased level of neurofibril tangles, increased tau phosphorylation, tau filament formation, abnormal neuronal morphology, lysosomal abnormalities, neuronal degeneration, and gliosis.

As used herein, “administering a treatment to an animal or cell” is intended to refer to dispensing, delivering or applying a treatment to an animal or cell. In terms of the therapeutic agent, the term “administering” is intended to refer to contacting or dispensing, delivering or applying the therapeutic agent to an animal by any suitable route for delivery of the therapeutic agent to the desired location in the animal, including delivery by either the parenteral or oral route, intramuscular injection, subcutaneous/intradermal injection, intravenous injection, buccal administration, transdermal delivery and administration by the intranasal or respiratory tract route. In certain embodiments, the animal or cell is an animal or cell described herein. In other embodiments, the method uses a transgenic mouse in which the expression of the PIN1 gene is inhibited. In yet other preferred embodiments, the method uses a cell derived from a transgenic mouse in which the expression of the PIN1 gene is inhibited. In still other embodiments, the animal is a human.

As used herein, the term “agent” includes any compound, e.g., peptides, peptidomimetics, nucleic acids, antibodies, small molecules, or other drugs, which ameliorate, delay or prevent a neurodegenerative disorder in a subject.

As used herein, the term “neurodegenerative disease or disorder” includes any disease disorder or condition that affects neuronal homeostasis, e.g., results in the degeneration or loss of neuronal cells. Neurodegenerative diseases include conditions in which the development of the neurons, i.e., motor or brain neurons, is abnormal, as well as conditions in which result in loss of normal neuron function. Examples of such neurodegenerative disorders include Alzheimer's disease, Pick disease, progressive supranuclear palsy, corticobasal degeneration, frontaltemporal dementia and parkinsonism linked to chromosome 17. Accordingly, a “Pin1 associated neurodegenerative disease or disorder” includes any neurodegenerative disease or disorder in which there is a decrease in the level of Pin1 protein or nucleic acid levels, Pin1 biological activity (e.g., isomerase activity). A decrease in Pin1 biological activity can be due to, for example, a decrease in protein levels, increase in Pin1 degradation, or an increase the phosphorylation of Pin1.

As used herein, the term “transgenic cell” refers to a cell containing a transgene.

As used herein, “purified preparation” is a preparation which includes at least 10%, more preferably 50%, yet more preferably 90% by number or weight of the subject cells.

As used herein, the term “increase the biological activity of Pin1” refers to a method in which a biological activity of Pin1, e.g., the isomerase activity, is increased. This can be accomplished by, for example, by contacting Pin1 with an agent that increases isomerase activity, by reducing Pin1 inhibitory phosphorylation, by increasing Pin1 expression or by increasing Pin1 protein stability.

The present invention is described in further detail in the following subsections.

I. Preparation of PIN1 Targeting Constructs

A. Knock-out Construct

The PIN1 nucleotide sequence to be used in producing the targeting construct is digested with a particular restriction enzyme selected to digest at a location(s) such that a new DNA sequence encoding a marker gene can be inserted in the proper position within this PIN1 nucleotide sequence. The marker gene should be inserted such that it can serve to prevent expression of the native gene. The position will depend on various factors such as the restriction sites in the sequence to be cut, and whether an exon sequence or a promoter sequence, or both is (are) to be interrupted (i.e., the precise location of insertion necessary to inhibit PIN1 gene expression). In some cases, it will be desirable to actually remove a portion or even all of one or more exons of the gene to be suppressed so as to keep the length of the targeting construct comparable to the original genomic sequence when the marker gene is inserted in the targeting construct. In these cases, the genomic DNA is cut with appropriate restriction endonucleases such that a fragment of the proper size can be removed.

The marker sequence can be any nucleotide sequence that is detectable and/or assayable. For example, the marker gene can be an antibiotic resistance gene or other gene whose expression in the genome can easily be detected. The marker gene can be linked to its own promoter or to another strong promoter from any source that will be active in the cell into which it is inserted; or it can be transcribed using the promoter of the PIN1 gene. The marker gene can also have a polyA sequence attached to the 3′ end of the gene; this sequence serves to terminate transcription of the gene. For example, the marker sequence can be a protein that (a) confers resistance to antibiotics or other toxins; e.g., ampicillin, tetracycline, or kanamycin for prokaryotic host cells, and neomycin, hygromycin, or methotrexate for mammalian cells; (b) complements auxotrophic deficiencies of the cell; or (c) supplies critical nutrients not available from complex media.

After the PIN1 DNA sequence has been digested with the appropriate restriction enzymes, the marker gene sequence is ligated into the PIN1 DNA sequence using methods known to the skilled artisan and described in Sambrook et al., Molecular Cloning A Laboratory Manual, 2nd Ed., ed., Cold Spring Harbor Laboratory Press: 1989, the contents of which are incorporated herein by reference.

Preferably, the ends of the DNA fragments to be ligated are compatible; this is accomplished by either restricting all fragments with enzymes that generate compatible ends, or by blunting the ends prior to ligation. Blunting is performed using methods known in the art, such as for example by the use of Klenow fragment (DNA polymerase I) to fill in sticky ends.

The ligated targeting construct can be inserted directly into embryonic stem cells, or it may first be placed into a suitable vector for amplification prior to insertion. Preferred vectors are those that are rapidly amplified in bacterial cells such as the pBluescript II SK vector (Stratagene, San Diego, Calif.) or pGEM7 (Promega Corp., Madison, Wis.).

B. Construct for Conditional Expression of Pin1

Conditional neuron-specific deletion of Pin1 can be generated using Cre- and loxP-mediated recombination using standard techniques. As the first step to reach this goal, mouse genomic BAC clones covering the Pin1 gene can be obtained from Incite Genetics. To generate the targeting vector, three Pin1 genomic fragments will be subcloned into the pflox vector, which consists of a selection marker PGK-Neo cassette flanked by two loxP sites and a third loxP site.

II. Construction of Transgenic Mice

A. Transfection of Embryonic Stem Cells

Mouse embryonic stem cells (ES cells) can be used to generate the transgenic (e.g., knockout) PIN1 mice. Any ES cell line that is capable of integrating into and becoming part of the germ line of a developing embryo, so as to create germ line transmission of the targeting construct is suitable for use herein. For example, a mouse strain that can be used for production of ES cells is the 129J strain. A preferred ES cell line is murine cell line D3 (American Type Culture Collection catalog no. CRL 1934). The cells can be cultured and prepared for DNA insertion using methods known in the art and described in Robertson, Teratocarcinomas and Embryonic Stem Cells: A Practical Approach, E. J. Robertson, ed. IRL Press, Washington, D.C., 1987, in Bradley et al., Current Topics in Devel. Biol., 20:357-371, 1986 and in Hogan et al., Manipulating the Mouse Embryo: A Laboratory Manual, Cold Spring Harbor Laboratory Press, Cold Spring Harbor, N.Y., 1986, the contents of which are incorporated herein by reference.

The knockout construct can be introduced into the ES cells by methods known in the art, e.g., those described in Sambrook et al. Suitable methods include electroporation, microinjection, and calcium phosphate treatment methods.

The targeting construct to be introduced into the ES cell is preferably linear. Linearization can be accomplished by digesting the DNA with a suitable restriction endonuclease selected to cut only within the vector sequence and not within the PIN1 gene sequence.

After the introduction of the targeting construct, the cells are screened for the presence of the construct. The cells can be screened using a variety of methods. Where the marker gene is an antibiotic resistance gene, the cells can be cultured in the presence of an otherwise lethal concentration of antibiotic. Those cells that survive have presumably integrated the knockout construct. A southern blot of the ES cell genomic DNA can also be used. If the marker gene is a gene that encodes an enzyme whose activity can be detected (e.g., beta-galactosidase), the enzyme substrate can be added to the cells under suitable conditions, and the enzymatic activity can be analyzed.

To identify those cells with proper integration of the targeting construct, the DNA can be extracted from the ES cells using standard methods. The DNA can then be probed on a southern blot with a probe or probes designed to hybridize in a specific pattern to genomic DNA digested with particular restriction enzymes. Alternatively, or additionally, the genomic DNA can be amplified by PCR with probes specifically designed to amplify DNA fragments of a particular size and sequence such that, only those cells containing the targeting construct in the proper position will generate DNA fragments of the proper size.

B. Injection/Implantation of Embryos

Procedures for embryo manipulation and microinjection are described in, for example, Manipulating the Mouse Embryo (Cold Spring Harbor Laboratory Press, Cold Spring Harbor, N.Y., 1986, the contents of which are incorporated herein by reference). Similar methods are used for production of other transgenic animals. In an exemplary embodiment, mouse zygotes are collected from six-week old females that have been super ovulated with pregnant mares serum (PMS) followed 48 hours later with human chorionic gonadotropin. Primed females are placed with males and checked for vaginal plugs on the following morning. Pseudo pregnant females are selected for estrus, placed with proven sterile vasectomized males and used as recipients. Zygotes are collected and cumulus cells removed. Furthermore, blastocytes can be harvested. Pronuclear embryos are recovered from female mice mated to males. Females are treated with pregnant mare serum, PMS, to induce follicular growth and human chorionic gonadotropin, hCG, to induce ovulation. Embryos are recovered in a Dulbecco's modified phosphate buffered saline (DPBS) and maintained in Dulbecco's modified essential medium (DMEM) supplemented with 10% fetal bovine serum.

Microinjection of a PIN1 targeting construct can be performed using standard micromanipulators attached to a microscope. For instance, embryos are typically held in 100 microliter drops of DPBS under oil while being microinjected. DNA solution is microinjected into the male pronucleus. Successful injection is monitored by swelling of the pronucleus. Recombinant ES cells can be injected into blastocytes, using similar techniques. Immediately after injection embryos are transferred to recipient females, e.g. mature mice mated to vasectomized male mice. In a general protocol, recipient females are anesthetized, paralumbar incisions are made to expose the oviducts, and the embryos are transformed into the ampullary region of the oviducts. The body wall is sutured and the skin closed with wound clips.

C. Screening for the Presence of the Targeting Construct

Transgenic (e.g., knockout) animals can be identified after birth by standard protocols. DNA from tail tissue can be screened for the presence of the targeting construct using southern blots and/or PCR. Offspring that appear to be mosaics are then crossed to each other if they are believed to carry the targeting construct in their germ line to generate homozygous knockout animals. If it is unclear whether the offspring will have germ line transmission, they can be crossed with a parental or other strain and the offspring screened for heterozygosity. The heterozygotes are identified by southern blots and/or PCR amplification of the DNA.

The heterozygotes can then be crossed with each other to generate homozygous transgenic offspring. Homozygotes may be identified by southern blotting of equivalent amounts of genomic DNA from mice that are the product of this cross, as well as mice that are known heterozygotes and wild type mice. Probes to screen the southern blots can be designed as set forth above.

Other means of identifying and characterizing the knockout offspring are known in the art. For example, northern blots can be used to probe the MRNA for the presence or absence of transcripts encoding the gene knocked out, the marker gene, or both. In addition, western blots can be used to assess the level of expression of the gene knocked out in various tissues of these offspring by probing the western blot with an antibody against the protein encoded by the gene knocked out (e.g., the PIN1 protein), or an antibody against the marker gene product, where this gene is expressed. Finally, in situ analysis (such as fixing the cells and labeling with antibody) and/or FACS (fluorescence activated cell sorting) analysis of various cells from the offspring can be performed using suitable antibodies to look for the presence or absence of the targeting construct gene product.

D. Mice Containing Neuron-Specific Deletion of Pin1

To delete Pin1 specifically in the brain neuron, Pin1/flox mice will be breed with mice carrying Cre recombinase under the control of the mouse Thy-1 promoter, as described (Dewachter et al., J. Neuronscience, 2002, 22:3445-3453). It has been shown that Thy-1 driven Cre expression becomes active in transgenic mice only in central neurons after birth. To confirm neuron-specific Pin1 deletion, immunoblotting and immunostaining analyses can be performed to make sure that no Pin1 protein is expressed specifically in the central neurons.

E. Mice Containing Multiple Mutations

Transgenic mice containing Pin1 mutations as described herein can be crossed with mice containing mutations in additional genes associated with neurodegenerative disorders. Mice that are heterozygous or homozygous for each of the mutations can be generated and maintained using standard crossbreeding procedures. Examples of mice that can be bred with mice containing Pin1 mutations include those that overexpress APP, tau and/or preselin (see references 59-63).

Alternatively, transgenic mice containing mutation in more than one gene, e.g., double knockout, can be generated using standard techniques such as those described herein.

III. Other Transgenic Animals

The transgenic animal used in the methods of the invention can be a mammal; a bird; a reptile or an amphibian. Suitable mammals for uses described herein include: ruminants; ungulates; domesticated mammals; and dairy animals. Preferred animals include: goats, sheep, camels, cows, pigs, horses, oxen, llamas, chickens, geese, and turkeys. Methods for the preparation and use of such animals are known in the art. A protocol for the production of a transgenic pig can be found in White and Yannoutsos, Current Topics in Complement Research: 64th Forum in Immunology, pp. 88-94; U.S. Pat. No. 5,523,226; U.S. Pat. No. 5,573,933; PCT Application WO93/25071; and PCT Application WO95/04744. A protocol for the production of a transgenic rat can be found in Bader and Ganten, Clinical and Experimental Pharmacology and Physiology, Supp. 3:S81-S87, 1996. A protocol for the production of a transgenic cow can be found in Transgenic Animal Technology, A Handbook, 1994, ed., Carl A. Pinkert, Academic Press, Inc. A protocol for the production of a transgenic sheep can be found in Transgenic Animal Technology, A Handbook, 1994, ed., Carl A. Pinkert, Academic Press, Inc.

IV. Candidate Compounds

Varieties of candidate compounds are known in the art and can be employed in the methods of the invention. Suitable compounds include those that increase the biological activity of Pin1 including, but not limited to, those that decrease the rate of Pin1 degradation of Pin1, decrease Pin1 phosphorylation, increase Pin1 catalytic activity, and/or increase Pin1 expression (e.g., by gene therapy). Such compounds can be identified by a number of art recognized assays such as those described herein.

For example, agents that increase the biological activity of Pin1 can be derived using Pin1 nucleic acid or amino acid sequences. The nucleotide and amino acid sequences of these molecules are known in the art and can be found in the literature or on a database such as GenBank. See, for example, Pin1 (Lu, K. P. et al. (1996) Nature. 380544-7 or GenBank Accession number AAC50492 or U49070).

A. Nucleic Acid Molecules

Nucleic acid molecules can also be used as modulators of Pin1 activity. In particular embodiments the nucleic acid molecules of the invention encode Pin1 or a biologically active portion of Pin1.

Given the sequences encoding Pin1 disclosed in the art, a nucleic acid for use in the methods of the invention can be constructed using chemical synthesis and enzymatic ligation reactions using procedures known in the art. For example, a nucleic acid molecule can be chemically or recombinantly synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

In yet another embodiment, the Pin1 nucleic acid molecules of the present invention can be modified at the base moiety, sugar moiety, or phosphate backbone to improve, e.g., the stability, hybridization, or solubility of the molecule. For example, the deoxyribose phosphate backbone of the nucleic acid molecules can be modified to generate peptide nucleic acids (see Hyrup, B. and Nielsen, P. E. (1996) Bioorg. Med. Chem. 4(1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup and Nielsen (1996) supra and Perry-O'Keefe et al. (1996) Proc. Natl. Acad. Sci. USA 93:14670-675.

Nucleic acid molecules of the invention can be produced by inserting the nucleic acid molecule into a vector and producing multiple copies of the vector and then isolating the nucleic acid sequence that encodes Pin1 or a portion of Pin1.

Gene therapy vectors are produced, for example, by using a viral vector, or transformed cell to introduce a nucleic acid into a cell. In order to practice the gene therapy methods are described in: U.S. patent application Ser. No. 2002/0,193,335 provides methods of delivering a gene therapy vector, or transformed cell, to neurological tissue; U.S. patent application Ser. No. 2002/0,187,951 provides methods for treating a neurodegenerative disease and/or symptoms thereof and/or preventing neurodegenerative disease and/or symptoms thereof, in a mammal by administering a lentiviral vector to a target cell in the brain or nervous system of the mammal; U.S. patent application Ser. No. 2002/0,107,213 discloses a gene therapy vehicle and methods for its use in the treatment and prevention of neurodegenerative disease; U.S. patent application Ser. No. 2003/0,099,671 discloses a mutated rabies virus suitable for delivering a gene to a subject; and U.S. Pat. No. 6,310,196 which describes a DNA construct which is useful for immunization or gene therapy; U.S. Pat. No. 6,436,708 discloses a gene delivery system which results in long-term expression throughout the brain has been developed; U.S. Pat. No. 6,140,111 which disclose retroviral vectors suitable for human gene therapy in the treatment of a variety of disease; and Kaspar B K et al. (2002) Mol Ther. 5:50-6, Suhr S T et al (1999) Arch Neurol. 56:287-92. Wong, P. C. et al. ((2002) Nat Neurosci 5, 633-639) describes neuronal specific promoters such as Thy1 which valuable in the practice of the methods of the instant invention due to the effects of aberrant Pin1 in non-neuronal tissues of the body.

B. Proteins and Peptides

In addition to the full length Pin1 polypeptide, a number of useful peptides can also be derived from Pin1 polypeptide sequences. A peptide may, for instance, be fragment of the naturally occurring protein, or a mimic or peptidomimetic of Pin1. Variants of Pin1 which can be generated by mutagenesis (e.g., amino acid substitution, amino acid insertion, or truncation of Pin1), and identified by screening combinatorial libraries of mutants, such as truncation mutants, of a Pin1 protein for the desired activity, (e.g., isomerase activity).

For example, a variegated library of Pin1 variants can be generated by combinatorial mutagenesis at the nucleic acid level, for example, by enzymatically ligating a mixture of synthetic oligonucleotides into gene sequences such that a degenerate set of potential Pin1 sequences is expressible as individual polypeptides, or alternatively, as a set of larger fusion proteins (e.g., for phage display) containing the set of Pin1 sequences therein. Chemical synthesis of a degenerate gene sequence can also be performed in an automatic DNA synthesizer, and the synthetic gene then ligated into an appropriate expression vector. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

Once suitable Pin1 polypeptides are identified, systematic substitution of one or more amino acids of the amino acid sequence, or a functional variant thereof, with a D-amino acid of the same type (e.g., D-lysine in place of L-lysine) can also be used to generate a peptide which has increased stability. In addition, constrained peptides comprising a Pin1 sequence, a functional variant thereof, or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

Peptides can be produced recombinantly or direct chemical synthesis. Further, peptides may be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments of the invention. Certain amino-terminal and/or carboxy-terminal modifications and/or peptide extensions to the core sequence can provide advantageous physical, chemical, biochemical, and pharmacological properties, such as: enhanced stability, -increased potency and/or efficacy, resistance to serum proteases, and desirable pharmacokinetic properties.

The invention further provides a peptide analog or peptide mimetic of the Pin1 protein. Peptide analogs are commonly used in the pharmaceutical industry as non-peptide drugs with properties analogous to those of the template peptide. These types of non-peptide compound are termed “peptide mimetics” or “peptidomimetics” (Fauchere, J. (1986) Adv. Drug Res. 15:29; Veber and Freidinger (1985) TINS p.392; and Evans et al. (1987) J. Med. Chem. 30:1229, which are incorporated herein by reference) and are usually developed with the aid of computerized molecular modeling. Peptide mimetics that are structurally similar to Pin1 or functional variants thereof can be used to produce an antagonistic effect. Generally, peptidomimetics are structurally similar to the paradigm polypeptide (Pin1) but have one or more peptide linkages optionally replaced by a linkage selected from the group consisting of: —CH2NH—, —CH2S—, —CH2-CH2—, —CH═CH— (cis and trans), —COCH2-, —CH(OH)CH2-, and —CH2SO—. This is accomplished by the skilled practitioner by methods known in the art which are further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH2NH—, CH2CH2-); Spatola, A. F. et al. (1986) Life Sci. 38:1243-1249 (—CH2-S); Hann, M. M. (1982) J. Chem. Soc. Perkin Trans. I. 307-314 (—CH—CH—, cis and trans); Almquist, R. G. et al. (190) J. Med. Chem. 23:1392-1398 (—COCH2-); Jennings-White, C. et al. (1982) Tetrahedron Lett. 23:2533 (—COCH2-); Szelke, M. et al. European Appln. EP 45665 (1982) CA: 97:39405 (1982) (—CH(OH)CH2-); Holladay, M. W. et al. (1983) Tetrahedron Lett. (1983) 24:4401-4404 (—C(OH)CH2-); and Hruby, V. J. (1982) Life Sci. (1982) 31:189-199 (—CH2-S—); each of which is incorporated herein by reference.

C. Small Molecules

Small molecules of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

D. Antibodies

In another embodiment, the invention employs antibodies to activate Pin1 function or stabilize Pin1, e.g, inhibit the degradation of Pin1. As used herein, the term “antibody” includes whole antibodies or antigen-binding fragments thereof including, for example, Fab, F(ab′)2, Fv and single chain Fv fragments. Suitable antibodies include any form of antibody, e.g., murine, human, chimeric, or humanized and any type antibody isotype, such as IgG1, IgG2, IgG3, IgG4, IgM, IgA1, IgA2, IgAsec, IgD, or IgE isotypes.

Antibodies which specifically bind Pin1, e.g., that result in conformational changes that stabilize Pin1 protein without inhibiting activity, or that activate the isomerase activity of Pin1, can serve as an agonist of Pin1. As used herein, “specific binding” refers to antibody binding to a predetermined antigen. Typically, the antibody binds with a dissociation constant (KD) of 10−7 M or less, and binds to the predetermined antigen with a KD that is at least two-fold less than its KD for binding to a non-specific antigen (e.g., BSA, casein) other than the predetermined antigen or a closely-related antigen. Several Pin1 antibodies are known, (see, for example, U.S. Pat. No. 6,596,848).

Alternatively, Pin1 antibodies can be produced according to well known methods for antibody production, and tested for agonist activity using the methods described herein. For example, antigenic peptides of Pin1 which are useful for the generation of antibodies can be identified in a variety of manners well known in the art. For example, useful epitopes can be predicted by analyzing the sequence of the protein using web-based predictive algorithms (BIMAS & SYFPEITHI) to generate potential antigenic peptides from which synthetic versions can be made and tested for their capacity to generate Pin1 specific antibodies.

The Pin1 antibodies can be monoclonal or polyclonal. The terms “monoclonal antibodies” as used herein, refers to a population of antibody molecules that contain only one species of an antigen binding site capable of immunoreacting with a particular epitope of an antigen, whereas the term “polyclonal antibodies” refers to a population of antibody molecules that contain multiple species of antigen binding sites capable of interacting with a particular antigen. Techniques for generating monoclonal and polyclonal antibodies are well known in the art (See, e.g., Current Protocols in Immunology, Coligan et al., eds., John Wiley & Sons, http://www.does.org/masterli/cpi.html).

Recombinant Pin1 antibodies, such as chimeric and humanized monoclonal antibodies, comprising both human and non-human portions can be made using standard recombinant DNA techniques, and are also within the scope of the invention. Such chimeric and humanized monoclonal antibodies can be produced by recombinant DNA techniques known in the art, for example using methods described in Robinson et al. International Patent Publication PCT/US86/02269; Akira, et al. European Patent Application 184,187; Taniguchi, M., European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl Cancer Inst. 80:1553-1559); Morrison, S. L. (1985) Science 229:1202-1207; Oi et al. (1986) BioTechniques 4:214; U.S. Pat. Nos. 5,225,539 5,565,332, 5,871,907, or 5,733,743; Jones et al. (1986) Nature 321:552-525; Verhoeyan et al. (1988) Science 239:1534; and Beidler et al. (1988) J. Immunol. 141:4053-4060.

Recombinant chimeric antibodies can be further humanized by replacing sequences of the Fv variable region which are not directly involved in antigen binding with equivalent sequences from human Fv variable regions. General reviews of humanized chimeric antibodies are provided by Morrison, S. L., 1985, Science 229:1202-1207 and by Oi et al., 1986, BioTechniques 4:214. Those methods include isolating, manipulating, and expressing the nucleic acid sequences that encode all or part of immunoglobulin Fv variable regions from at least one of a heavy or light chain. Sources of such nucleic acid are well known to those skilled in the art. The recombinant DNA encoding the chimeric antibody, or fragment thereof, can then be cloned into an appropriate expression vector. Suitable humanized antibodies can alternatively be produced by CDR substitution U.S. Pat. No. 5,225,539; Jones et al. 1986 Nature 321:552-525; Verhoeyan et al. 1988 Science 239:1534; and Beidler et al. 1988 J. Immunol. 141:4053-4060.

Fully human antibodies that bind to Pin1 can also be employed in the invention, and can produced using techniques that are known in the art. For example, transgenic mice can be made using standard methods, e.g., according to Hogan, et al., “Manipulating the Mouse Embryo: A Laboratory Manual”, Cold Spring Harbor Laboratory, which is incorporated herein by reference, or are purchased commercially. Embryonic stem cells are manipulated according to published procedures (Teratocarcinomas and embryonic stem cells: a practical approach, Robertson, E. J. ed., IRL Press, Washington, D.C., 1987; Zjilstra et al. (1989) Nature 342:435-438; and Schwartzberg et al. (1989) Science 246:799-803, each of which is incorporated herein by reference). For example, transgenic mice can be immunized using purified or recombinant Pin1 or a fusion protein comprising at least an immunogenic portion of Pin1. Antibody reactivity can be measured using standard methods. The term “recombinant human antibody,” as used herein, includes all human antibodies that are prepared, expressed, created or isolated by recombinant means. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Single chain antagonistic antibodies that bind to Pin1 or their respective ligand or receptor also can be identified and isolated by screening a combinatorial library of human immunoglobulin sequences displayed on M13 bacteriophage (Winter et al. 1994 Annu. Rev. Immunol. 1994 12:433; Hoogenboom et al., 1998, Immunotechnology 4: 1).

In yet another embodiment of the invention, bispecific or multispecific antibodies that bind to Pin1 or antigen-binding portions thereof. Such antibodies can be generated, for example, by linking one antibody or antigen-binding portion (e.g., by chemical coupling, genetic fusion, noncovalent association or otherwise) to a second antibody or antigen-binding portion. Bispecific and multispecific molecules of the present invention can be made using chemical techniques, “polydoma” techniques or recombinant DNA techniques. Bispecific and multispecific molecules can also be single chain molecules or may comprise at least two single chain molecules. Methods for preparing bi- and multispecific molecules are described for example in D. M. Kranz et al. (1981) Proc. Natl. Acad. Sci. USA 78:5807; U.S. Pat. No. 4,474,893; U.S. Pat. No. 5,260,203; U.S. Pat. 5,534,254. U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858.

Also within the scope of the invention are chimeric and humanized antibodies in which specific amino acids have been substituted, deleted or added. In particular, preferred humanized antibodies have amino acid substitutions in the framework region, such as to improve binding to the antigen. For example, in a humanized antibody having mouse CDRs, amino acids located in the human framework region can be replaced with the amino acids located at the corresponding positions in the mouse antibody. Such substitutions are known to improve binding of humanized antibodies to the antigen in some instances. Antibodies in which amino acids have been added, deleted, or substituted are referred to herein as modified antibodies or altered antibodies.

The term modified antibody is also intended to include antibodies, such as monoclonal antibodies, chimeric antibodies, and humanized antibodies which have been modified by, e.g., deleting, adding, or substituting portions of the antibody. For example, an antibody can be modified by deleting the constant region and replacing it with a constant region meant to increase half-life, e.g., serum half-life, stability or affinity of the antibody. Any modification is within the scope of the invention so long as the bispecific and multispecific molecule has at least one antigen binding region specific for an FcR and triggers at least one effector function.

E. Other Molecules

Pin1 levels have been shown to be decreased upon prolonged exposure to the microtubule-targeting drug Taxol, which can apparently be prevented by some proteasome inhibitors (Basu, et al. (2002) Neoplasia 4, 218-227). Accordingly, such proteasome inhibitors can be combined with any of the candidate compounds described herein to decrease the degradation of Pin1.

Further, it has been demonstrated that Pin1 function can be inhibited by phosphorylation, and that Pin1 phosphorylation can be induced by activation of PKA (Lu, et al., J. Biol. Chem. 277:2381-2384). Accordingly, inhibitors of PKA or other Pin1 kinases, or alternatively, Pin1 phosphatases can also be used alone or in combination with any of the candidate compounds described herein to decrease Pin1 phosphorylation, and thus, increase Pin1 isomerase activity.

V. Screening Assays

The invention provides a method (also referred to herein as a “screening assay”) for testing candidate compounds or agents (as described above) which ameliorate, prevent or delay one or more neurodegenerative phenotypes associated with a neurodegenerative disorder.

The invention provides in vivo and in vitro methods of identifying agents that are capable of being used in the methods of the invention.

A. In Vitro Methods

In certain embodiments, the candidate compounds are first examined in vitro in a cell-based assay comprising contacting a cell expressing of PIN1 (e.g., a decreased level) with a test compound and determining the ability of the test compound to modulate (e.g., stimulate) the activity of the PIN1 target molecule. Cell based assays useful for examining Pin1 activity are well-known in the art, and include those described in the Examples set forth below, and also can found, for example, U.S. Pat. Nos. 6,258,582, 6,462,173B1, 6,495,376, U.S. patent application Ser. No. 2002/025,521, and Fisher et al. (Biomed. Biochim. Acta, 1984, 43: 1101-1111), the entire contents each of which are expressly incorporated herein by reference.

In particular embodiments, the cell is a neuronal cell, e.g., the established neuronal cell line PC12 derived from rat pheochromocytoma. In other embodiments, the cell can be derived from the animal models described herein. For example, in one embodiment, the ability of the test compound to modulate the activity of a PIN1 target molecule can be accomplished by determining the ability of the PIN1 protein to bind to or interact with the PIN1 target molecule, e.g., Tau.

In further embodiments, the ability of a compound to decrease Pin1 protein degradation, or to decrease Pin1 phosphorylation can be tested using methods described, for example, in Basu, et al. 2002) Neoplasia 4, 218-227, and Lu, et al., J. Biol. Chem. 277:2381-2384.

B. In Vivo Methods

The animal model of neurodegenerative disease described herein can be used to further test the candidate compounds identified using the in vitro methods of the invention. PIN1 misexpressing animals, e.g., mice, or cells can be used to screen for treatments for PIN1-related disorders, e.g., neurodegenerative disorders. The candidate treatment can be administered over a range of doses to the animal or cell. Efficacy can be assayed at various time points for the effects of the compound on the treatment or prevention of the disorder being evaluated. For example, use of compounds for the treatment or prevention of a neurodegenerative condition includes treatment of the animal to, thereby identify treatments suitable for administration to human subjects.

Such treatments can be evaluated by determining the effect of the treatment on the onset, progression or reversal of a neurodegenerative phenotype. Such parameters include age-dependent phenotypes such as a reduction in mobility, a reduction in vocalization, abnormal limb-clasping reflex, retinal atrophy inability to succeed in a hang test, an increased level of MPM-2 epitopes, an increased level of neurofibril tangles, increased tau phosphorylation, neuronal degeneration, and gliosis. Methods for identifying and monitoring neurodegenerative phenotypes can be accomplished using standard, well-known methods as described in the foregoing examples.

VI. Candidate Treatments

The candidate treatment, which is evaluated using methods described herein, can include: (a) the administration of a therapeutic agent (e.g., a drug, a chemical, an antibody, a protein, a nucleic acid or other substance) to a PIN1 misexpressing animal or cell; (b) the administration of a diet regimen to an PIN1 misexpressing animal; (c) the administration of ionizing radiation to an PIN1 misexpressing animal or cell. Any combination of the aforementioned treatments can be administered to a PIN1 misexpressing animal or cell. The treatment can be administered prior to, simultaneously and/or after the onset of the disorder or condition, for which the candidate treatment is being evaluated. The therapeutic agent can be administered to a PIN1 misexpressing animal, orally, parenterally or topically.

VII. Pharmaceutical Compositions

In certain embodiments, the method involves administering an agent (e.g., an agent identified by a screening assay described herein), or combination of agents that ameliorates, prevents, or delays the onset or progression of a neurodegenerative disorder as measured by the effect on one or more neurodegenerative phenotypes associated with the disorder.

The PIN1 nucleic acid molecules, Pin1 proteins, fragments of PIN1 proteins, small molecules, and anti-PIN1 antibodies of the invention can be incorporated into pharmaceutical compositions suitable for administration. Such compositions typically comprise the nucleic acid molecule, protein, small molecule or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intracranial, intraspinal, intradermal, intracranial, intraspinal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a PIN1 protein or peptide, a gene therapy vector containing a PIN1 nucleic acid or an anti-PIN1 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In particular embodiments, the compounds of the invention can be formulated to ensure proper distribution in vivo. For example, the blood-brain barrier (BBB) excludes many highly hydrophilic compounds. To ensure that the compounds of the invention cross the BBB, they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134), different species of which may comprise the formulations of the inventions, as well as components of the invented molecules; p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273. In one embodiment of the invention, the therapeutic compounds of the invention are formulated in liposomes; in a more preferred embodiment, the liposomes include a targeting moiety. In a most preferred embodiment, the therapeutic compounds in the liposomes are delivered by bolus injection to a site proximal to the desired area.

In other embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma, or neuronal fluid (e.g., spinal fluid) concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a kit, e.g., container, pack, or dispenser together with instructions for administration.

VIII. Therapeutic Methods

Pin1 was shown to bind to phosphorylated Tau and amyloid precursor protein peptides in U.S. Pat. No. 6,495,376 (the entire contents of which are incorporated herein by reference). The data presented in the instant examples demonstrates for the first time that depletion of Pin1 or Pin1 activity can result in the onset of neurodegenerative disease. Accordingly, the present invention includes to methods of treating, preventing or delaying the onset or progression of a neurodegenerative disorder by administering an agent identified according to the screening methods of the invention. Agents that can be used in the methods of the invention include a nucleic acid or a protein, an antibody, a peptidomimetic, antisense nucleic acid molecules, or other small molecules as identified by the methods described herein. Further, the agents identified by the methods disclosed herein can be tested, e.g., for efficacy and toxicity, in an animal model of neurodegenerative disease disclosed herein. These modulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by aberrant neuronal development or maintenance, e.g., a neurodegenerative disorder.

In one aspect, the invention provides a method of treating subject suffering from a neurodegenerative disease or disorder characterized by a decrease in Pin1 wherein a subject is administered an agent that increases the biological activity of Pin1. In one particular embodiment the instant invention provides a combination method wherein a subject is subjected to a diagnostic method of the invention and if it is found that there is a decrease in Pin1 biological activity, the subject is administered a compound that increases the biological activity of Pin1.

In another aspect, the invention provides a method of treating a subject having a neurodegenerative disorder comprising administering to the subject an agent identified in the mouse model of the instant invention that increases Pin1 biological activity.

A neuropathological hallmark in Alzheimer's disease is the presence of increased levels of phosphorylated Tau in neuronal tissue or fluids. Tau levels can be measured in a subject by obtaining a sample of spinal fluid or brain, e.g., a biopsy. Accordingly, in another embodiment, the invention provides a method of treating a subject that has increased level of Tau in a biological sample with an agent that increases the biological activity of Pin1. In one aspect, Tau can be phosphorylated at position 231 (Tau231P) and detected using the methods described in WO 02/04949. Further, Tau can be measured by the methods described by Voorheis, et al. in U.S. patent application Ser. No. 2002/0,002,270.

In another aspect, the invention provides a method for preventing in a subject, neurodegenerative disease, by administering to the subject an agent that increases the biological activity of Pin1. Subjects at risk for a disease which is caused or contributed to by aberrant Pin1 expression or activity can be identified by, for example, any or a combination of diagnostic or prognostic assays as described herein. Administration of a prophylactic agent can occur prior to the manifestation of symptoms characteristic of the Pin1 aberrancy, such that a disease or disorder is prevented or, alternatively, delayed in its progression. Depending on the type of Pin1 aberrancy, for example, a Pin1 protein, Pin1 agonist or Pin1 antagonist agent can be used for treating the subject. The appropriate agent can be determined based on screening assays described herein.

In one aspect of the invention, gene delivery is used to treat a subject having, or at risk for having, a neurodegenerative disorder. Gene delivery is accomplished using gene therapy methods in which nucleic acid encoding Pin1 is provided to the nervous system such that Pin1 biological activity is increased. In one particular aspect, a nucleic acid molecule introduced using gene therapy can be any biologically active portion of Pin1, e.g., the isomerase domain.

Pharmaceutical compositions used in the methods of invention can be delivered to a subject by, for example, intravenous injection, intraspinal injection, intracranial injection, local administration (see U.S. Pat. No. 5,328,470) or by stereotactic injection (see e.g., Chen et al. (1994) Proc. Natl. Acad. Sci. USA 91:3054-3057). In certain embodiments, methods of the invention can include administration of intact recombinant cells that produce biologically active Pin1, e.g., expressed by a gene delivery vector as described herein.

IX. Diagnostic Methods

In another aspect of the invention, a diagnostic method is provided in which a biological sample, e.g., brain tissue or spinal fluid, is isolated from a subject and assayed for the presence of Pin1. A decrease in the level of Pin1 protein or activity in a sample relative to a control is indicative of a Pin1-associated neurodegenerative condition that can be treated by methods of the invention using agents identified in the methods of the invention. Further, a decrease in the level of Pin1 in a sample relative to a control is indicative that a subject would benefit from treatment with an agent that increases the biological activity of Pin1.

In one embodiment, the level of total Pin1 protein in the sample is determined. In an alternative embodiment, the level biological activity of Pin1 is determined. In still another embodiment, the level of phosphorylated Pin1 is measured. Biological samples for testing can be obtained using standard techniques, e.g., by syringe or biopsy (e.g., needle biopsy). Assays including immunoassays and the like that are useful for examining Pin1 protein levels and/or activity are well-known in the art, and are described, for example, in the Examples set forth below. See also, U.S. Pat. Nos. 6,258,582, 6,462,173B1, 6,495,376, U.S. patent application Ser. No. 2002/025,521, PCT /US02/03697, PCT/US02/03658, Fisher et al. (Biomed. Biochim. Acta, 1984, 43: 1101-1111), and Lu et al. (J. Biol. Chem. 277(4) 2381-2384, 2002), the entire contents each of which are expressly incorporated herein by reference.

This invention is further illustrated by the following examples which should not be construed as limiting. The contents of all references, patents and published patent applications cited throughout this application are incorporated herein by reference.

### EXAMPLES

Methods

Human samples and Pin1^{-/-} mouse strains.

Paraffin-fixed human samples of the hippocampus and parietal cortex were obtained from the University of Kentucky Alzheimer's Disease Research Center and consisted of 10 AD cases and 8 controls, with mean ages at death being 78±9 years and 79±9 years, respectively. The mean postmortem intervals were 2.8 hours for AD brains and 2.9 hours for controls. All AD subjects met the clinical and neuropathologic NIA-RI criteria for AD. Control subjects had no evidence of neurological disorders. The genetic background of Pin1^{-/-} mice is mixed 129/Sv and C57L/B622. Young and old mice were 2-3 and 9-14 months old, respectively. All the results have been reproduced in multiple animals.

Immunhistochemistry and Immunofluorescent Staining.

Conventional immunocytochemistry on human samples was performed using affinity-purified Pin1 polyclonal antibodies and mAb, as described (18). For double labeling experiments, sections were first stained with anti-Pin1 antibodies, and then stained with AT8, which was visualized with the Nickel intensifying method. For immunofluorescent staining, sections were incubated with affinity-purified anti-Pin1 antibodies and AT8 mAb, which were detected by Cy3- and FITC-conjugated secondary antibodies. For mouse tissues, deparaffinized or floating brain sections were immunostained with various antibodies using an ABC kit (Vector Labs) or Cy3-conjugated secondary antibodies (22).

Immunoblotting Analysis.

Sarcosyl-insoluble extracts were carried out as described (7, 8, 10). Briefly, brain tissues were homogenized in a buffer containing 10 mM Tris-HCl (pH 7.4), 0.8 M NaCl, 1 mM EGTA and 10% sucrose. After centrifugation, the supernatants were brought to 1% N-lauroylsacosinate and sarcosyl-insoluble extracts collected by centrifugation, followed by immunoblotting analysis 18. Alz50, MC1, TG3 and PHF-1 tau mAbs were kindly provided by Dr. P. Davies at Albert Einstein College of Medicine. AT8 and AT180, Tau-5 were purchased from Innogenetics and Biosource. The specificity of tau antibodies were: Tau-5 specific to both phosphorylated and non-phosphorylated tau; PHF-1 to pSer396 and pSer404; AT8 to pSer199 and pSer202; AT180 to pThr231; TG3 to pThr231 in a NFT-specific conformation; Alz50 and MC1 to NFT-specific conformations.

Hang Test, Neuron Count and Nissl Staining.

These assays were performed, as described (5, 6). For counting neurons, coronal sections at different regions of brains from age-matched Pin1^{-/-} and WT littermates were processed in parallel for NeuN staining. The inner layer of the parietal cortex (S1BF) at the level of 1.8 mm posterior to Bregma was selected and neuron counts performed at comparable areas of each mouse, and an average number of two adjacent fields were obtained for each region of each mouse. For the spinal cord, the numbers of large neurons per anterior horn were counted and an average of two nearby sections was calculated for each mouse.

Kinase and Phosphatase Assays.

Total kinase activity in brain lysates was assayed by autophosphorylation in the presence of Mg2+ and g[32P]-ATP, while CDKs and GSK-β3 activity towards tau was assayed after purification using p13suc1 beads and anti-GSK-β3 antibodies, respectively, as described¹⁸. Phosphatase PP2A activity towards the phosphopeptide RRApTVA (Promega) was assayed as described²⁴. Phosphatase activity towards tau that was phosphorylated by Cdc2 was also assayed as described (18, 19).

Gallyas Silver Staining and Thioflavin-S Staining.

Gallyas silver staining and thioflavin-S staining were performed as described (6-10).

EM and immunogold-EM. To observe tau filaments, sarcosyl-insoluble extracts isolated from Pin1^{-/-} and control mouse brains were resuspended and placed on carbon-coated grids and stained with phosphotungstic acid, followed by EM and immunogold-EM using AT8 and AT180 as described (7, 10).

Results

Pin1-catalyzed prolyl isomerization can regulate the function and/or dephosphorylation of certain phosphoproteins, many of which are also recognized by the mitosis- and phosphorylation-specific antibody (mAb) MPM-2. Interestingly, induction of MPM-2 epitopes is a prominent common feature of AD, frontotemporal dementia with Parkinsonism linked to chromosome 17 (FTDP-17), Down Syndrome, corticobasal degeneration, progressive supranuclear palsy and Pick's disease (13, 14). In fact, tau phosphorylation pattern in AD is similar to that in mitotic cells (12, 14). Together with reduction of soluble Pin1 in late stages of AD brains (18), we thus hypothesized that Pin1 might protect against neurodegeneration. However, Holzer et al. reported that in AD hippocampus Pin1 expression was primarily found in a small group of tangle-free degenerative neurons in CA1 and CA2, but not in CA3 and CA4 non-degenerative neurons and proposed that Pin1 promoted neurodegeneration (20).

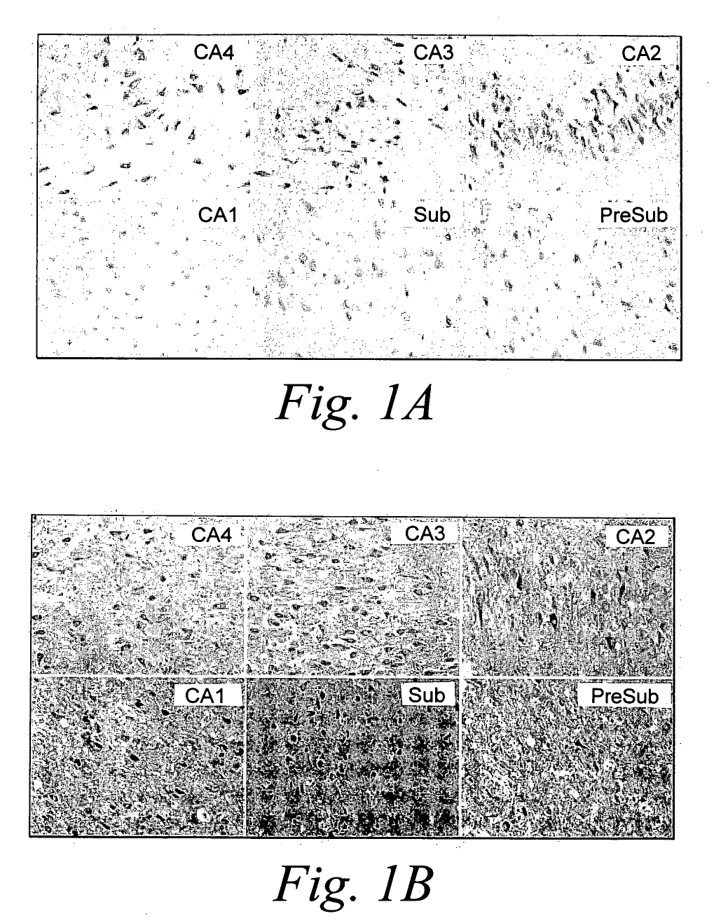

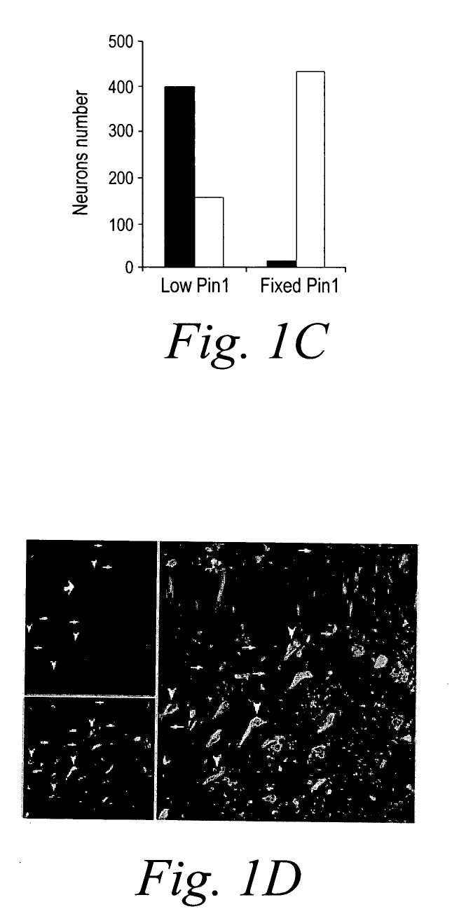

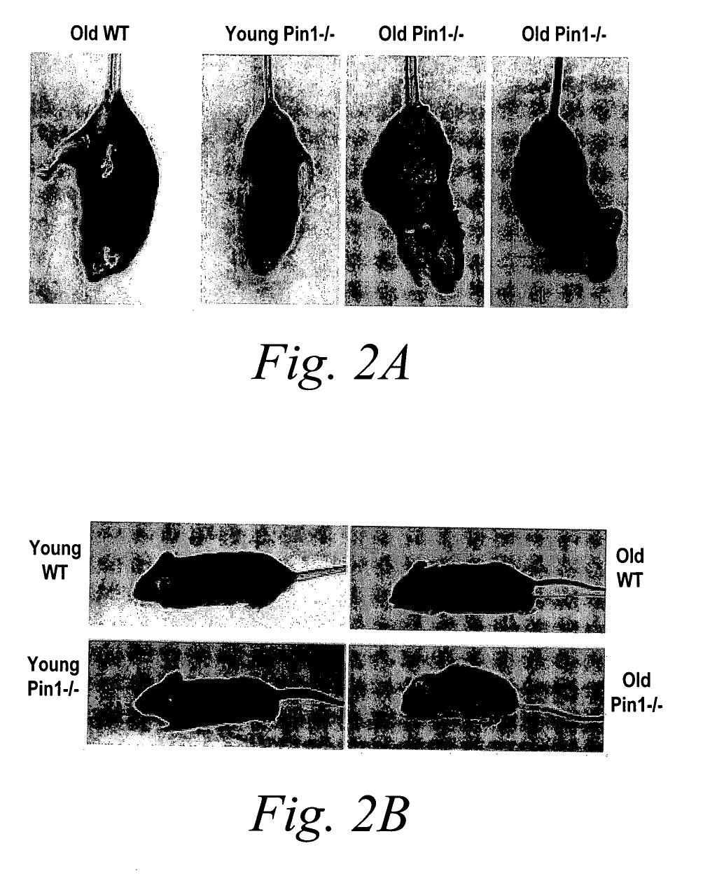

Neurons in different subregions of the hippocampus and neocortex are known to have differential vulnerability to neurofibrillary degeneration in AD (21). To examine the relationship between this predicted vulnerability and Pin1 expression, we examined Pin1 expression in normal human hippocampus and parietal cortex using immunostaining. Pin1 was detected in the cytoplasm in addition to the nucleus of neurons (FIG. 1). Interestingly, Pin1 expression displayed an obvious subregional difference in all brain sections from 8 normal cases (FIG. 1a). In the hippocampus, Pin1 expression was relatively higher in CA4, CA3, CA2 and presubiculum, but lower in CA1 and subiculum. In the parietal cortex, Pin1 expression was relatively higher in layer IIIb-c neurons, but lower in layer V neurons. Those subregions containing low Pin1 expression are known to be prone to, whereas those containing high Pin1 expression are spared from, neurofibrillary degeneration in AD, suggesting an inverse correlation between Pin1 expression and the predicted vulnerability.

To extend this correlation, we immunostained human 10 AD brain sections doubly with anti-Pin1 antibodies, and an AT8 antibody that detects early neurofibrillary degeneration. Tangle-bearing neurons were enriched in CA1 and subiculum of the hippocampus and layer V of the parietal cortex (FIG. 1b). We also observed that very high Pin1 immunoreactivity was detected at cytoplasmic granules in a small number of neurons mainly in CA1, which is likely due to sequestration of Pin1 by strong MPM-2 epitopes in these granules (20). However, in contrast to that reported (20), we found that Pin1 expression was readily detected in CA3 and CA4 subregions and in fact was higher than that in tangle-rich subregions (FIG. 1b). Overall, 96% of pyramidal neurons that contained relatively higher Pin1 did not have tangles, whereas 71% of neurons that contained relatively lower Pin1 had tangles (FIG. 1c). Even within the tangle-prone CA1 and subiculum of the hippocampus, Pin1 expression in most tangle-bearing neurons was still lower than that in tangle-free neurons (FIG. 1d). Furthermore, in the CA1 subregion adjacent to CA1, Pin expression in most tangle-free neurons was higher than in tangle-containing neurons. Pin1 expression was also relatively higher in tangle-sparing layer IIIb-c neurons, but lower in tangle-rich layer V neurons in parietal cortex.

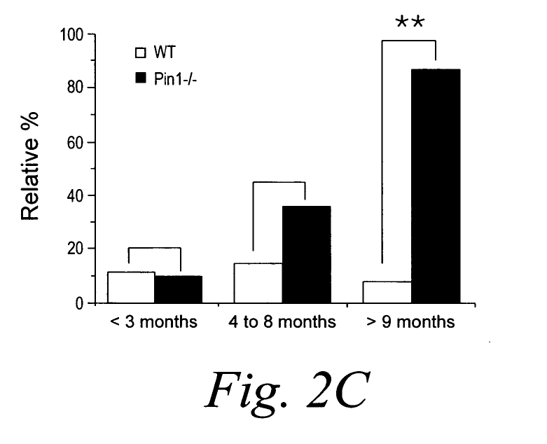

These results indicate an inverse correlation between Pin1 expression and actual neurofibrillary degeneration in AD, and indicate that Pin1 can protect against neurodegeneration. To test this idea, we examined neuronal phenotypes of Pin1^{-/-} mice. These mice do develop several age-dependent phenotypes, including retinal atrophy (22). Since retinal atrophy can be a feature of neurodegeneration, Pin1^{-/-} mice might exhibit other neuronal phenotypes. Indeed, Pin1^{-/-} mice but not their WT littermates displayed progressive age-dependent motor and behavioral deficits, which included abnormal limb-clasping reflexes (FIG. 2a), hunched postures (FIG. 2b), reduced mobility and eye irritation. When subjected to a hang test⁶, most old, but not young Pin1^{-/-} mice fell after grasping the rope only briefly (FIG. 2c). Thus, Pin1^{-/-} mice develop progressive age-dependent motor and behavioral deficits, as do tau transgenic mice (6, 10).

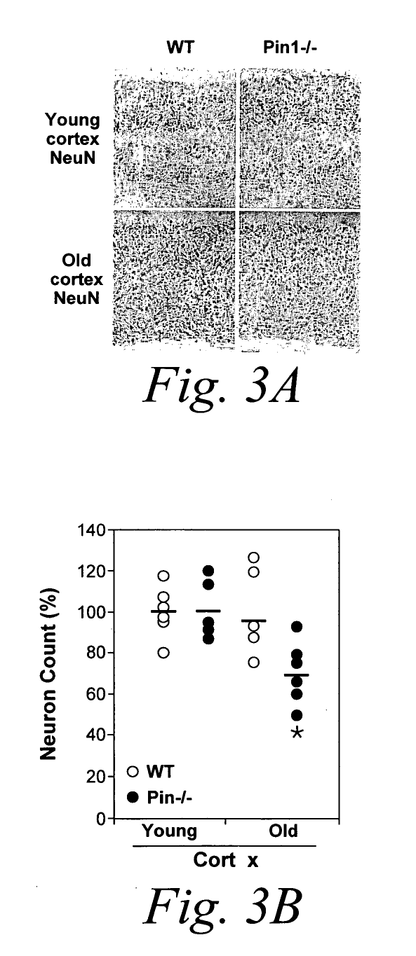

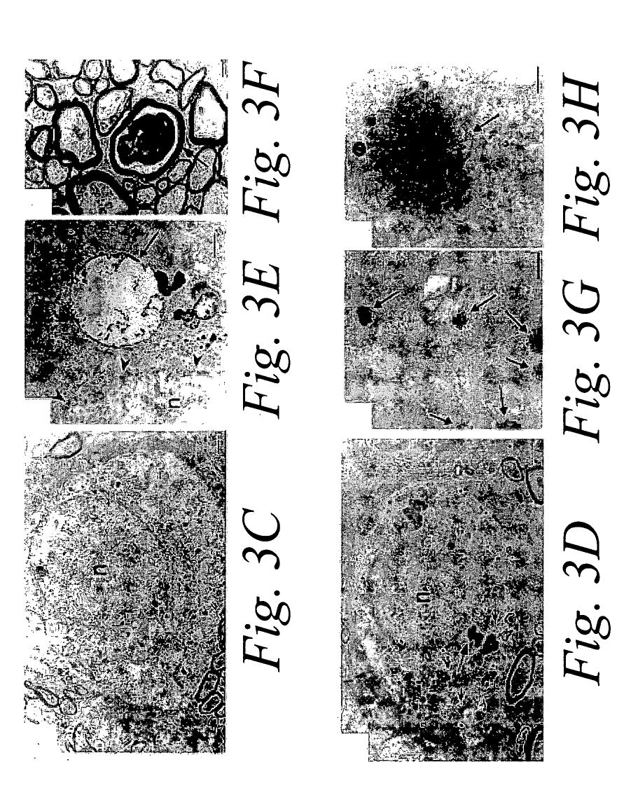

We next examined if Pin1^{-/-} mice had any age-dependent neuronal loss. The number of NeuN-positive neurons was significantly decreased in the parietal cortex of old, but not young Pin1^{-/-} mice (FIGS. 3a, b). A similar neuronal loss was also found in spinal cord at the cervical and lumbar regions of Pin1^{-/-} mice. Nissl staining also confirmed neuronal degeneration, as shown by abnormally stained cytoarchitecture of Pin1^{-/-} neurons. Some Pin1^{-/-} neurons had swollen cell bodies, as observed in tau transgenic mice (10). However, no obvious neuronal loss was found in some other brain regions notably cerebellum, although there was Pin1 expression. These results show that loss of Pin1 causes age-dependent neuronal loss in certain regions of the central nervous system.

To confirm degeneration, we performed electron microscopy (EM) (e.g., of parietal cortex, hippocampus and spinal cord). In contrast to WT neurons (FIG. 3c), many Pin1^{-/-} neurons showed dark, degenerating granules or organelles adjacent to the nucleus (FIG. 3d), and often had autophagic vacuoles (FIG. 3e), consistent with degenerated lysosomes. Degeneration was also observed in some axons (FIG. 3f). Another notable change found mostly in Pin1^{-/-} neuronal processes, but not in WT controls, was the presence of electron-dense structures that consisted of compact and radiating filament-like structures without other visible organelles or vesicles (FIGS. 3g, h). These ultrastructural changes confirm neurodegeneration in Pin1^{-/-} neurons.

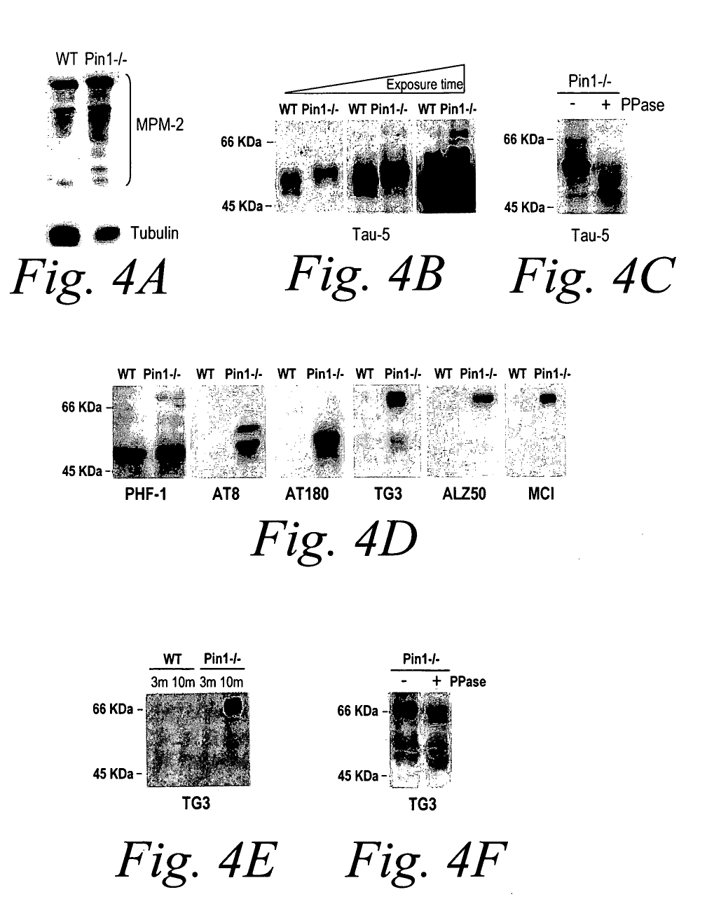

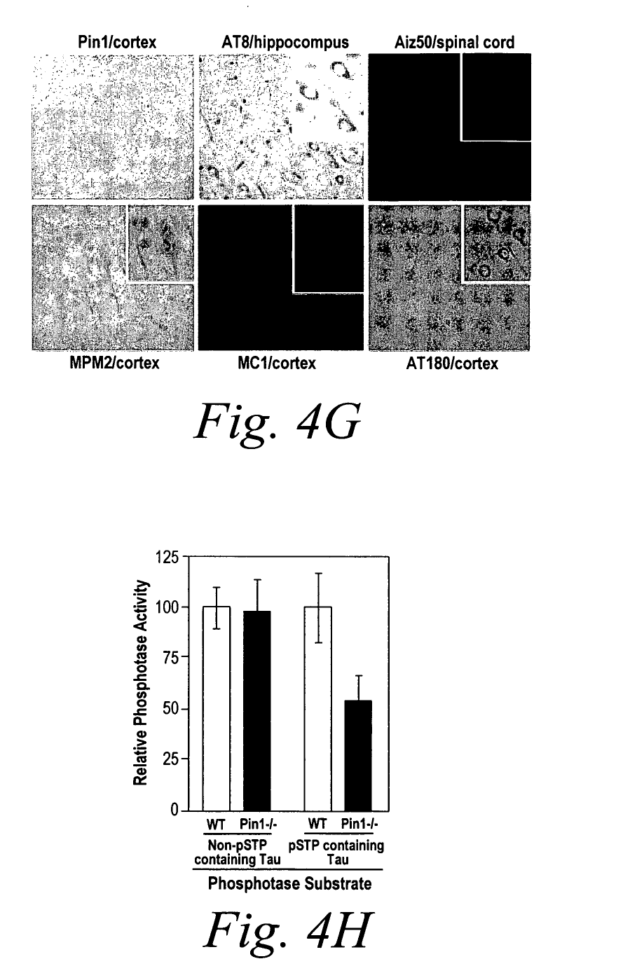

The findings that Pin1^{-/-} mice develop neuronal degeneration in an age-dependent manner in certain brain regions indicate that the effects of Pin1 loss are specific. The ability of Pin1 to promote dephosphorylation of MPM-2 epitopes also suggested that Pin1 knockout could lead to an accumulation of MPM-2 epitopes, an early common characteristic of AD and related disorders^{12, 14, 23}. Indeed, total MPM-2 reactivity was ˜3-fold higher in Pin1^{-/-} brain lysates than in controls (FIG. 4a), which was confirmed by immunostaining (FIG. 4g). In contrast, induction of other cell cycle markers including cyclin D1, CdK4, Ki67 and phosphorylated histone H3 was not detected. These results indicate that Pin1 loss or absence (e.g., in knockout mice) leads to neuronal induction of MPM-2 epitopes.

To examine the mechanisms underlying the neurodegeneration, we then focused on tau because tau-related pathologies are a hallmark of AD and other tauopathies^{2,3}, and have been well-characterized in mice (5-10). Importantly, tau-related pathologies are similar to those in Pin1^{-/-} mice. Furthermore, tau is a major MPM-2 antigen and a well-characterized Pin1 substrate (18, 19). Pin1 specifically acts on the pThr231-Pro motif in tau and induces a conformational change, thereby restoring tau function and promoting tau dephosphorylation by PP2A because of the phosphatase conformation specificity (18, 19). Reduction of PP2A activity also increases tau phosphorylation in mice (24). Thus, we hypothesized that tau would be aberrantly phosphorylated and exhibit abnormal conformations in Pin1^{-/-} mice.

To examine this hypothesis, we isolated sarcosyl-insoluble extracts from brains of Pin1^{-/-} and WT mice, followed by immunoblotting analysis (FIGS. 4b-f). All sarcosyl insoluble tau isoforms from Pin1^{-/-} mice had much slower mobility on SDS gels than WT controls (FIG. 4b), indicating an increase in total tau phosphorylation. This was confirmed by an increase in mobility upon phosphatase treatment, and immunobloting with phospho-specific tau mAbs (FIGS. 4c,d). AT8 and AT180 strongly recognized the slower migrating tau isoforms, whereas TG3 selectively recognized the slowest migrating tau isoforms (˜68 kDa), which was also recognized by PHF-1 (FIG. 4d). Moreover, this TG3 epitope was induced in an age-dependent manner and phosphatase treatment significantly, although not completely, reduced the TG3 signal (FIGS. 4e,f). Finally, 68 kDa tau in Pin1^{-/-} brain was also strongly recognized by Alz50 and MC1 (FIG. 4d), which detect neurofibrillary tangle (NFT)-specific conformations (26, 27). These results indicate that 68kDa tau in Pin1^{-/-} brain is hyperphosphorylated and contains NFT conformations. Of note, tau in NFTs of AD is notoriously resistant to complete dephosphorylation (25), and there is a similar 68 kDa tau isoform (A68) in human AD that is the defining component of PHFs and is also recognized by Alz50 (26, 27). Immunostaining also showed strong immunoreactivities with AT180, AT8, MC1 and Alz50 in the somatodendritic region of Pin1^{-/-}, but not WT neurons in the parietal cortex, brainstem, hippocampus, and spinal cord (FIG. 4g). The presence of pTau in the cytoplasm and axons of Pin1^{-/-}, but not WT neurons was confirmed by immuno-gold EM (FIGS. 3c, d, S6). These results indicate that the absence of Pin1 in the knockout mouse causes age-dependent tau hyperphosphorylation and NFT-specific conformations.

To explore mechanisms for induction of MPM-2 and tau phosphoepitopes, we compared kinase and phosphatase activity in Pin1^{-/-} and WT brains. Although there was no significant increase in the activity of total kinases, Cdks or GSK-β3 (FIGS. S7a, b), we found a significant decrease in phosphatase PP2A activity towards pSer/Thr-Pro motifs in tau, but not towards a non-pSer/Thr-Pro phosphopeptide, in Pin1^{-/-} brain lysates before obvious neurodegeneration (FIG. 4h). These results indicate that Pin1 specifically affects dephosphorylation of pSer/Thr-Pro motifs, consistent with that Pin1 is required for PP2A to dephosphorylate tau (19).

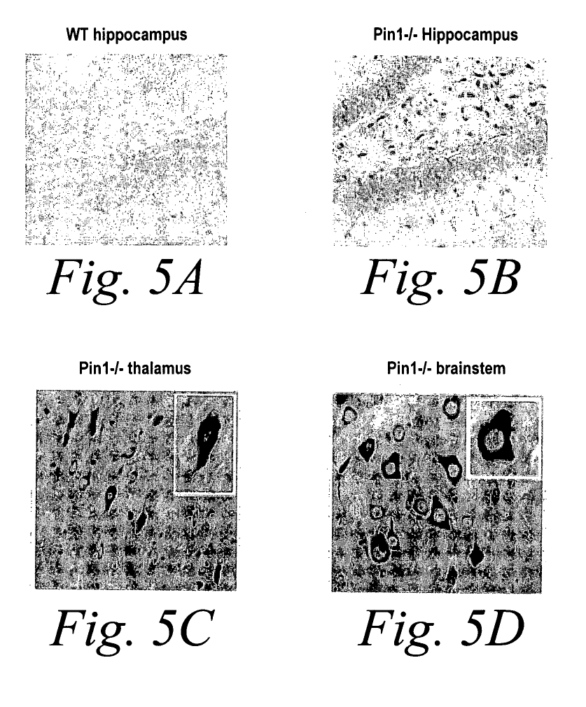

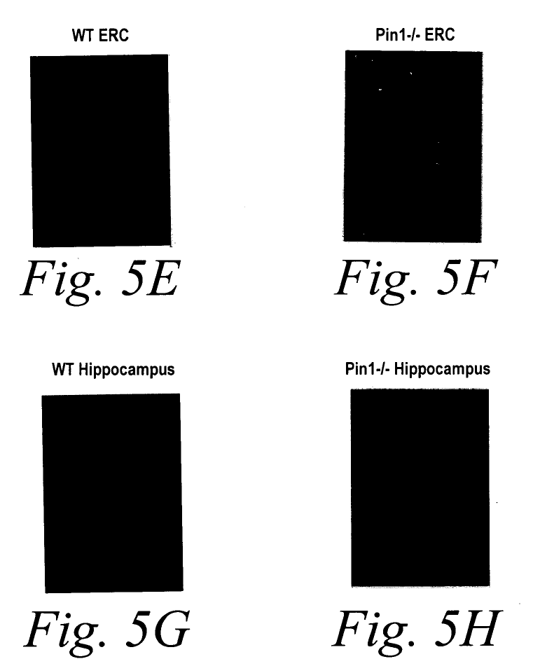

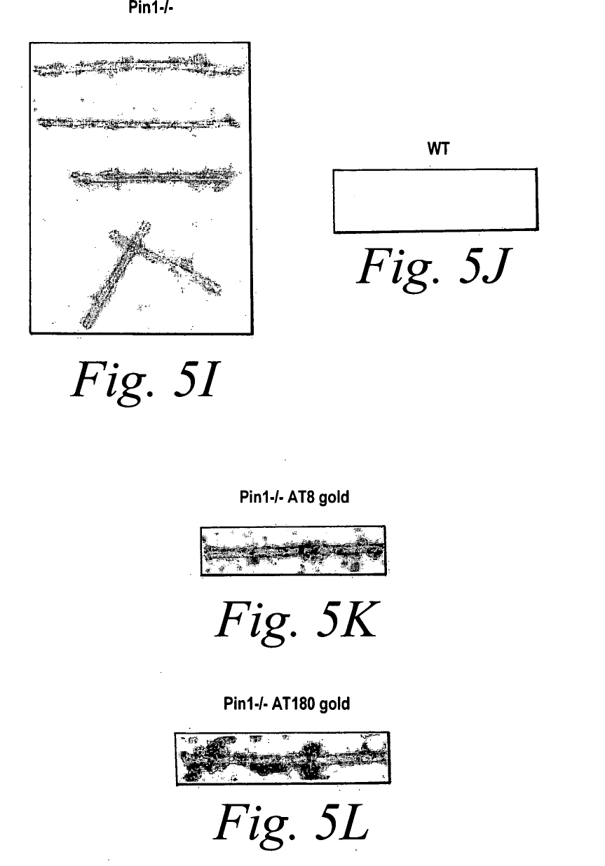

The findings that Pin1^{-/-} neurons are strongly positive with phospho- or NFT-specific tau mAbs indicates that they contain tau filaments. To confirm this, we first used Gallyas silver staining and thioflavin-S staining, which have been used to detect NFTs (6, 9, 29). In Pin1^{-/-} mice (FIGS. 5b-d), but not in WT controls (FIG. 5a), we observed some Gallyas silver-positive neurons in the hippocampus, thalamus and brainstem. Furthermore, a small fraction of Pin1^{-/-}, but not WT, neurons in the hippocampus, spinal cord and especially entorhinal cortex were also positive for thioflavin-S staining (FIGS. 5e-h), with a pattern similar to that in tau transgenic mice (10). These results show that Pin1^{-/-} neurons display NFT-like pathologies.

To confirm the formation of tau filaments, we subjected sarcosyl-insoluble tau isolated from brain extracts to EM and immuno-gold EM. In extracts from 8 out of 12 old Pin1^{-/-} mice but not WT brains, we readily found filaments that were twisted or straight and ˜15 nm wide (FIGS. 5i, j). Further immuno-gold EM showed that the filaments were labeled with AT8 and AT180 (FIGS. 5k, l), but not with anti-tubulin mAB, confirming that they are tau filaments. These results demonstrate that loss of Pin1 function causes the formation of endogenous tau filaments in mice.

In summary, we have shown that Pin1 expression inversely correlates with neuronal vulnerability in normal brain, and also with neurofibrillary degeneration in AD brain. Furthermore, Pin1^{-/-} mice develop age-dependent neuropathy, characterized clinically by motor and behavioral deficits and pathologically by tau hyperphosphorylation, tau filament formation and neuronal loss in brain and spinal cord. Interestingly, all these neuronal phenotypes are strikingly similar to those induced by transgenic overexpression of tau or its mutants (5-10). These results provide the first genetic evidence for a critical role of Pin1 in protecting against age-dependent neurodegeneration. Furthermore, this is the first clear demonstration that endogenous mouse tau can form tau filaments. Therefore, while overexpression of APP, presenilin and tau elicits AD and/or tau-related pathologies, Pin1 is the first protein whose deletion causes age-dependent neurodegeneration and tau pathologies. Moreover, the demonstration that Pin1-mediated post-phosphorylation regulation plays a pivotal role in the maintenance of normal neuronal function underscores the role of protein phosphorylation in neurodegenerative diseases and offers new insight into the pathogenesis and treatment of AD and other tauopathies.

Given the phenotypic similarity between Pin1^{-/-} and tau transgenic mice, tau-related pathologies likely play an important role in Pin1^{-/-} induced neurodegeneration, although other Pin1-related deficits also contribute to this process²². Manipulation of either tau kinases or phosphatases has been shown to increase pTau in mice (14, 24, 29, 30). We have now shown that loss of Pin1 in neurons reduces phosphatase activity specifically towards pSer/Thr-Pro motifs and induces tau hyperphosphorylation, NFT conformations and tau filament formation. These results indicate that tau is normally regulated by dynamic phosphorylation and dephosphorylation. If Pin1 function is low as in the CA1 region or absent as in Pin1^{-/-} mice, certain pSer/Thr-Pro motifs in pTau might not be isomerized resulting in their existence in aberrant conformations. Since TG3 immunoreactivity towards the pThr231-Pro tau peptide can be affected by TFE, a solvent use to increase cis content of molecules, the aberrant pThr231-Pro conformation of tau peptides in Pin1^{-/-} mice is likely in Cis conformation consistent with the lack of Pin1 isomerase activity. As a result, pTau cannot be dephosphorylated and/or functionally restored and instead aggregates into tau filaments, eventually contributing to neurodegeneration. This is consistent with the inverse correlation between Pin1 levels and neurofibrillary degeneration in AD and with the difficulty to dephosphorylate tau in NFTs of AD (25) and in the insoluble faction of Pin1⁻ mice. This also provides an explanation for the findings that although manipulating tau kinase or phosphatase activities can induce tau phosphorylation and some pre-tangle pathologies, it is not sufficient to induce tau filament and neuronal loss in mice (14, 24, 29), unless rapidly induced at high levels (30). As a result, pTau cannot be properly dephosphorylated and/or functionally restored, leading to tau hyperphosphorylation, NFT formation and neurodegeneration.

### References

1. Selkoe, D. J. The cell biology of beta-amyloid precursor protein and presenilin in Alzheimer's disease. Trends Cell Biol 8, 447-453 (1998).

2. Mandelkow, E. M. & Mandelkow, E. Tau in Alzheimer's disease. Trends Cell Biol 8, 425-427 (1998).

3. Lee, V. M., Goedert, M. & Trojanowski, J. Q. Neurodegenerative tauopathies. Annu Rev Neurosci 24, 1121-1159 (2001).

4. Wong, P. C., Cai, H., Borchelt, D. R. & Price, D. L. Genetically engineered mouse models of neurodegenerative diseases. Nat Neurosci 5, 633-639 (2002).

5. Ishihara, T., et al. Age-dependent emergence and progression of a tauopathy in transgenic mice overexpressing the shortest human tau isoform. Neuron 24, 751-762 (1999).

6. Lewis, J., et al. Neurofibrillary tangles, amyotrophy and progressive motor disturbance in mice expressing mutant (P301L) tau protein. Nat Genet 25, 402-405 (2000).

7. Gotz, J., Chen, F., Barmettler, R. & Nitsch, R. M. Tau filament formation in transgenic mice expressing P301L tau. J Biol Chem 276, 529-534 (2001).

8. Lewis, J., et al. Enhanced neurofibrillary degeneration in transgenic mice expressing mutant tau and APP. Science 293, 1487-1491 (2001).

9. Gotz, J., Chen, F., van Dorpe, J. & Nitsch, R. M. Formation of neurofibrillary tangles in P3011 tau transgenic mice induced by Abeta 42 fibrils. Science 293, 1491-1495 (2001).

10. Allen, B., et al. Abundant tau filaments and nonapoptotic neurodegeneration in transgenic mice expressing human P301S tau protein. J Neurosci 22, 9340-9351 (2002).

11. Bancher, C., et al. Accumulation of abnormally phosphorylated tau precedes the formation of neurofibrillary tangles in Alzheimer's disease. Brain Res 477, 90-99 (1989).

12. Preuss, U. & Mandelkow, E. M. Mitotic phosphorylation of tau protein in neuronal cell lines resembles phosphorylation in Alzheimer's disease. Eur J Cell Biol 76, 176-184 (1998).

13. Vincent, I., Zheng, J. H., Dickson, D. W., Kress, Y. & Davies, P. Mitotic phosphoepitopes precede paired helical filaments in Alzheimer's disease. Neurobiol Aging 19, 287-296 (1998).

14. Lu, K. P., Liou, Y. C. & Vincent, I. Proline-directed phosphorylation and isomerization in mitotic regulation and in Alzheimer's disease. BioEssays 25, 174-181 (2003).

15. Lu, K. P., Hanes, S. D. & Hunter, T. A human peptidyl-prolyl isomerase essential for regulation of mitosis. Nature 380, 544-547 (1996).

16. Yaffe, M. B., et al. Sequence-specific and phosphorylation-dependent proline isomerization: A potential mitotic regulatory mechanism. Science 278, 1957-1960 (1997).

17. Lu, K. P., Liou, Y. C. & Zhou, X. Z. Pinning down the proline-directed phosphorylation signaling. Trends Cell Biol 12, 164-172 (2002).

18. Lu, P. J., Wulf, G., Zhou, X. Z., Davies, P. & Lu, K. P. The prolyl isomerase Pin1 restores the function of Alzheimer-associated phosphorylated tau protein. Nature 399, 784-788 (1999).

19. Zhou, X. Z., et al. Pinl-dependent prolyl isomerization regulates dephosphorylation of Cdc25C and tau proteins. Mol Cell 6, 873-883 (2000).

20. Holzer, M., et al. Inverse association of Pin1 and tau accumulation in Alzheimer's disease hippocampus. Acta Neuropathol (Berl) 104, 471-481 (2002).

21. Davies, D. C., Horwood, N., Isaacs, S. L. & Mann, D. M. The effect of age and Alzheimer's disease on pyramidal neuron density in the individual fields of the hippocampal formation. Acta Neuropathol (Berl) 83, 510-517 (1992).

22. Liou, Y. C., et al. Loss of Pin1 function in the mouse resembles the cyclin D1-null phenotypes. Proc. Natl. Acad. Sci. USA 99, 1335-1340 (2002).

23. Husseman, J. W., Nochlin, D. & Vincent, I. Mitotic activation: a convergent mechanism for a cohort of neurodegenerative diseases. Neurobiol Aging 21, 815-828 (2000).

24. Kins, S., et al. Reduced protein phosphatase 2A activity induces hyperphosphorylation and altered compartmentalization of tau in transgenic mice. J Biol Chem 276, 38193-38200 (2001).

25. Gordon-Krajcer, W., Yang, L. & Ksiezak-Reding, H. Conformation of paired helical filaments blocks dephosphorylation of epitopes shared with fetal tau except Ser199/202 and Ser202/Thr205. Brain Res 856, 163-175 (2000).

26. Wolozin, B. L., Pruchnicki, A., Dickson, D. W. & Davies, P. A neuronal antigen in the brains of Alzheimer patients. Science 232, 648-650 (1986).

27. Lee, V. M., Balin, B. J., Otvos, L., Jr. & Trojanowski, J. Q. A68: a major subunit of paired helical filaments and derivatized forms of normal Tau. Science 251, 675-678 (1991).

28. Jicha, G. A., et al. A conformation- and phosphorylation-dependent antibody recognizing the paired helical filaments of Alzheimer's disease. J Neurochem 69, 2087-2095 (1997).

29. Patrick, G. N., et al. Conversion of p35 to p25 deregulates Cdk5 activity and promotes neurodegeneration. Nature 402, 615-622 (1999).

30. Lucas, J. J., et al. Decreased nuclear beta-catenin, tau hyperphosphorylation and neurodegeneration in GSK-β3 conditional transgenic mice. Embo J 20, 27-39 (2001).

1. A method for identifying an agent useful for the treatment of a neurodegenerative disorder comprising the steps of:
(a) administering an agent to a animal comprising a mutation in a Pin1 gene, wherein the animal displays a neurodegenerative phenotype associated with the mutation; and
(b) monitoring the neurodegenerative phenotype in the animal, wherein a decrease in the severity of the neurodegenerative phenotype is indicative that the agent is useful for treating a neurodegenerative disorder.

2. A method for identifying an agent useful for preventing or delaying the onset of a neurodegenerative disorder comprising the steps of:
(a) administering an agent to a animal comprising a mutation in a Pin1 gene; and
(b) monitoring the animal for a neurodegenerative phenotype associated with the mutation, wherein the absence or delay in the onset of the phenotype in the animal is indicative that the agent is useful for preventing or delaying onset of the neurodegenerative disorder.

3. A method for identifying an agent useful for preventing or delaying the progression of a neurodegenerative disorder comprising the steps of:
(a) administering an agent to a animal comprising a mutation in a Pin1 gene; and
(b) monitoring the animal for a neurodegenerative phenotype associated with the mutation, wherein the absence or delay in the progression of the neurodegenerative phenotype is indicative that the agent is useful for preventing or delaying the progression of the neurodegenerative disorder.

4. The method of claims 1, 2 or 3 wherein the neurodegenerative phenotype is an age-dependent neurological phenotype.

5. The method of claims 1, 2 or 3 wherein the neurodegenerative phenotype is a reduction in mobility.

6. The method of claims 1, 2 or 3 wherein the neurodegenerative phenotype is a reduction in vocalization.

7. The method of claims 1, 2 or 3 wherein neurodegenerative phenotype is abnormal limb-clasping reflex.

8. The method of claims 1, 2 or 3 wherein the neurodegenerative phenotype is retinal atrophy.

9. The method of claims 1, 2 or 3 wherein the neurodegenerative phenotype is an inability to succeed in a hang test.

10. The method of claims 1, 2 or 3 wherein the neurodegenerative phenotype is an increased level of MPM-2.

11. The method of claims 1, 2 or 3 wherein the neurodegenerative phenotype is an increased level of neurofibril tangles.

12. The method of claims 1, 2 or 3 wherein the neurodegenerative phenotype is increased tau phosphorylation.

13. The method of claims 1, 2 or 3 wherein the neurodegenerative phenotype is an increased level of tau filament formation.

14. The method of claims 1, 2 or 3 wherein the neurodegenerative phenotype is an increased level of abnormal neuronal morphology.

15. The method of claims 1, 2 or 3 wherein the neurodegenerative phenotype is an increased level of lysosomal abnormalities.

16. The method of claims 1, 2 or 3 neurodegenerative phenotype is neuronal degeneration.

17. The method of claims 1, 2 or 3 wherein the neurodegenerative phenotype is gliosis.

18. The method of claims 1, 2 or 3 wherein the Pin1 mutation disrupts the expression of the Pin1 gene.

19. The method of claim 18, wherein the Pin1 mutation is homozygous.

20. The method of claims 1, 2 or 3, wherein the Pin1 mutation is a conditional mutation that disrupts expression of the Pin1 gene in brain neurons.

21. The method of claim 20, wherein the Pin 1 mutation is heterozygous.

22. The method of claim 20, wherein the Pin1 mutation is homozygous.

23. The method of claims 1, 2 or 3 wherein the further comprises a mutation is a second gene associated with a neurodegenerative phenotype.

24. The method of claim 23, wherein the mutation results in the overexpression of APP.

25. The method of claim 23, wherein the mutation results in the overexpression of tau.

26. The method of claim 23, wherein the mutation results in the overexpression of preselin.

27. The method of claims 1, 2 or 3, wherein the animal further comprises a second mutation in one or more genes selected from the group consisting the genes encoding APP, tau and preselin.

28. A method of treating a subject with a neurodegenerative disorder comprising the step of administering the agent of claim 1 to the subject such that the subject is treated.

29. A method of preventing or delaying the onset of a neurodegenerative disorder comprising the step of administering the agent of claim 2 to the subject such that the onset of said neurodegenerative disorder is delayed or prevented.

30. A method of preventing or delaying the progression of a neurodegenerative disorder comprising the step of administering the agent of claim 3 to the subject such that the progression of said neurodegenerative disease is delayed or prevented.

31. The method of claims 28, 29 or 30, wherein the neurodegenerative disorder is selected from the group consisting of Alzheimer's disease, Pick disease, progressive supranuclear palsy, corticobasal degeneration, frontaltemporal dementia and parkinsonism linked to chromosome 17.

32. The method of claims 28, 29 or 30, wherein the neurodegenerative disorder is characterized by retinal atrophy.

33. The method of claims 28, 29 or 30, wherein the neurodegenerative disorder is characterized by a reduction in mobility.

34. The method of claims 28, 29 or 30, wherein the neurodegenerative disorder is characterized by neuronal degeneration.

35. The method of claims 25, 26, or 27, wherein the neurodegenerative disorder is characterized by tauopathy.

36. A method of evaluating the efficacy of a treatment for a neurodegenerative disorder comprising:
administering the treatment to a animal comprising a mutation in a Pin1 gene or a cell therefrom, wherein the animal displays a neurodegenerative phenotype associated with the mutation; and
determining the effect of the treatment on the neurodegenerative phenotype, thereby evaluating the efficacy of the treatment.

37. A method of treating a subject having a neurodegenerative disease associated with a decrease in the level of Pin1 comprising:
administering to said subject an effective amount of an agent that increases the biological activity of Pin1;
thereby treating said subject.

38. The method of claim 37 wherein said agent is a small molecule.

39. The method of claim 37 wherein said agent is a peptide.

40. The method of claim 37 wherein said agent is a gene therapy vector.

41. The method of claim 40 wherein said gene therapy vector is under the control of a neuronal specific promoter.

42. The method of claim 41 wherein said promoter is the Thy-1 promoter.

43. The method of claim 40 wherein said gene therapy vector encodes the isomerase domain of Pin1.

44. The method of claim 37 wherein the biological activity of Pin1 is increased by stabilizing Pin1.

45. The method of claim 37 wherein Pin1 is stabilized by reducing Pin1 inhibitory phosphorylation.

46. A method of treating a subject with a Pin1 associated neurodegenerative disease, comprising
(a) identifying a subject having a Pin1 associated neurodegenerative disease, and
(b) administering to the subject an effective amount of agent that increases the biological activity of Pin1.

47. The method of claim 46, wherein the subject is identified by determining the level of phosphorylated Tau in a biological sample, wherein an increased level of phosphorylated Tau in the sample is indicative of a Pin1-associated neurodegenerative disease.
